(19) United States
(12) Patent Application Publication
(10) Pub. No.: US 2009/0042735 A1
(43) Pub. Date: Feb. 12, 2009

(54) Methods and Compositions Related to Nucleic Acid Detection

(76) Inventors: Steven M. Blair, Salt Lake City, UT (US); Alexander M. Chagovetz, Salt Lake City, UT (US); Justin Bishop, West Jordan, UT (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/042,516

(22) Filed: Mar. 5, 2008

Related U.S. Application Data
(60) Provisional application No. 60/893,029, filed on Mar. 5, 2007.

(51) Int. Cl. C40B 30/04; C12Q 1/68

(52) U.S. Cl. 506/9; 435/6

(57) ABSTRACT

Disclosed are compositions and a method for detection of nucleic acid sequences based on competitive displacement.

### CROSS-REFERENCE TO RELATED APPLICATIONS

This application claims the benefit of U.S. provisional application No. 60/893,029 filed on Mar. 5, 2007. The aforementioned application is herein incorporated by this reference in its entirety.

### BACKGROUND OF THE INVENTION

The disclosed invention is in the general field of nucleic acid detection, and specifically in the field of detection of nucleic acids through competitor-based methods.

The mitochondrial genome is comprised of circular double stranded DNA of 16.6 Kbp in size, similar to other known plasmid DNAs. This genome mainly encodes for enzymes of the respiratory chain and transport RNAs (tRNA), and is characterized by a relatively high rates of point mutations (Wong 2004). SNP analysis of mtDNA is used therefore in forensic science, evolutionary genetics, and, lately, for molecular diagnostic purposes. In the latter case, some ensembles of mtDNA SNPs, mapped to key enzymes of ATP synthesis cascade and tRNA genes, were correlated with several hereditary neuro-muscular and neuro-degenerative syndromes (Wong 2004; Wong 2005). Analysis of the relative abundance of these pathological SNPs in the total population of mtDNA (heteroplasmy analysis) is of particular interest for diagnostic and prognostic purposes. Indeed, low abundance of heteroplasmy (less than 5%) is characteristic of a negative diagnosis for pathologies in question, while high abundance (over 50%) is severely symptomatic. It is therefore imperative to develop quantitative heteroplasmy analysis for diagnostic applications.

Currently, there are several methodologies applied to heteroplasmy analysis: direct sequencing of mtDNA, SNP-specific nuclease-denaturing HPLC (Wave DHPLC), and single nucleotide extension-capillary electrophoresis (SNE-CE). Fourier transform mass spectrometry (FT-MS) is also another example of emerging technologies targeting heteroplasmy analysis. Direct sequencing of mtDNA is the gold standard in heteroplasmy analysis; however, it is still prohibitively expensive and time consuming for practical clinical implementation and it is difficult to obtain quantitative heteroplasmy assessment.

Nuclease mismatch specific treatment is the basis of Wave technology: PCR amplified targets are mixed with reference dsDNA and allowed to form heteroduplexes through a melting-annealing cycle. Resulting dsDNA is then subjected to nuclease treatment specific for cleaving dsDNA at mismatched positions. Nuclease digest is then applied to denaturing HPLC where DNA fragments are identified based on size and stabilities of the duplexes (Elkano 2007). DHPLC can provide semi-quantitative answers, but for heteroplasmy analysis, it requires using two different reference sequences for heteroduplex formation in a sequential manner, or resolving multiple (more than 2) fragments during DHPLC analysis. Additionally, it involves several steps of sample processing (hybridizations and nuclease treatments) which are time consuming and expensive in clinical applications.

Single nucleotide extension is a proven method for SNP detection and quantitation: PCR products are annealed to different size-distinguishable primers adjacent to the loci of interest, which are subsequently extended by DNA polymerase in the presence of fluorescently labeled dideoxynucleotides (terminators) (Rudy 2006). The reaction mixture is then subjected to capillary electrophoresis, where extended primers are sized and particular dideoxynucleotides are indicative of the complementary base in the loci of interest. SNE-CE can provide quantitative results required for heteroplasmy analysis; however, it is labor intensive and parallel analysis of several loci is complicated from an assay design standpoint—14 different primers have to be easily distinguished by size. Alternatively, sequential analysis of multiple loci becomes cost and time prohibitive.

FT-MS technology is a direct method for SNP detection in PCR products, and does not require fluorescent labeling of the samples or additional enzymatic steps. This approach is based on analysis of amplified targets using sizing by mass, with subsequent quantitative analysis based on Fourier transform of mass spectra. There have been some encouraging results recently with application of FT-MS to heteroplasmy analysis (Hall 2005; Jiang 2007; Oberhauer 2007). However, there are several features which limit implementation of FT-MS in the clinical environment: capital equipment cost, sequential mode of operation, and several purification steps required for processing target DNA for MS analysis. There is another inherent disadvantage of this approach: although accuracy of MS analysis is adequate to detect SNPs in short PCR amplicons, SNP position is not interrogated, so one is necessarily faced with the assumption that the observed point mutation is assigned to the position of interest, which leaves unacceptable uncertainty in diagnostic reports.

DNA microarrays can interrogate multitudes of SNPs in a highly parallel fashion, thereby providing a rapid, cost-effective diagnostic solution. Although some microarray-based technologies have shown reliable SNP detection (for example, Illumina bead-chip technology), successes in quantitative SNP analysis have been limited: current quantitative microarray experiments are based on two color ratiometric approach, which is ill-suited for SNP analysis. This methodology, where analysis is performed by comparing fluorescence intensities from a reference sample to an “unknown” sample, is based on the assumptions of equal qualitative composition of the samples, which may not be true for heteroplasmy analysis, and the reaction may not reach equilibrium.

There are several technical limitations when applying direct microarray-based methods to SNP diagnostics: inconsistency in substrate surface coating, variability of probe attachment during spotting, and low coupling efficiencies of nucleotides during in situ oligonucleotide synthesis. These problems have been addressed lately by several microarray providers (e.g. Agilent, NimbleGen, Affymetrix). More fundamental problems arise from inherent limitations in surface capture, two of which we have studied: incomplete surface capture reactions and limited dynamic range of discrimination among highly homologous targets (for example SNPs). The latter problem has traditionally been addressed by combining surface capture with mismatch-specific ligation reaction, where the substrate specificity of ligase dramatically (3-5 orders of magnitude) improves the dynamic range of discrimination due to fidelity of mismatch recognition (Li 2006; Lee 2005). A significant disadvantage of ligation approaches is that they require the additional step of enzymatic treatment, where the ligase activity may be attenuated by proximity to the surface. Moreover, in order to achieve quantitative results, different allele-specific probes have to be introduced sequentially in different reactions, complicating analysis.

The importance of reaching equilibrium to obtain stable quantitative readings from the hybridization spots on an array is well recognized (Bhanot 2003). It was also convincingly demonstrated by experimental and theoretical studies that under standard hybridization conditions (i.e. 16-24 hours of hybridization at 45-60° C.), equilibrium is typically not reached for sub-nM concentrations (Dai 2002). As discussed further below, the mechanism of selectivity in molecular recognition is competitive displacement, which necessitates that equilibrium be reached when end-point detection is performed.

In recent publications, the mechanisms of SNP discrimination in surface capture were studied (Zhang 2005; Bishop 2006; Home 2006). It is established now that molecular recognition utilizes the mechanism of competitive displacement of lower affinity species by higher affinity species in the transient (kinetic) regime. As surface reactions move to equilibrium, the relative abundance of bound high affinity species grows, while the abundance of lower affinity species is described by non-monotonic behavior: in the beginning of the reaction the bound concentration grows, but later it decreases. This non-monotonic behavior is a function of the concentrations of different species and their corresponding rate constants for association and dissociation.

Current technologies are either labor intensive (restriction polymorphism, single nucleotide extension) or require significant capital equipment investments (FT-MS, Illumina-based genotyping) and often do not provide accuracy required in the reference laboratory environment

It is therefore an object of the present invention to provide a method and compositions for detecting nucleic acid sequence with a combination of specificity and sensitivity.

It is another object of the disclosed invention to detect nucleic acid sequences while discriminating between closely related sequences.

It is another object of the present invention to provide a method and compositions for detecting the amount and location of nucleic acid sequences with a combination of specificity and sensitivity.

### BRIEF SUMMARY OF THE INVENTION

Disclosed are compositions and methods of determining sequence similarity of a target nucleic acid and a probe, the method comprising: bringing into contact the target nucleic acid, the probe, and a labeled competitor nucleic acid; simultaneously incubating the target nucleic acid, a probe, and the labeled competitor nucleic acid under conditions suitable for hybridization; determining the binding pattern of the competitor nucleic acid to the probe in the presence of the target nucleic acid; and determining sequence similarity of the target nucleic acid and the probe based on the results of the previous step.

Also disclosed herein are methods of detecting the presence of a target nucleic acid in a sample, the method comprising: simultaneously bringing into contact the sample, a probe, and a labeled competitor nucleic acid; incubating the sample, the probe, and the labeled competitor nucleic acid under conditions suitable for hybridization; determining the binding pattern of the competitor nucleic acid to the probe in the presence of the sample; and detecting the presence of the target nucleic acid based on the results of the previous step.

Disclosed herein is a method of quantifying a target nucleic acid, the method comprising: simultaneously bringing into contact a target nucleic acid, a probe, and a labeled competitor nucleic acid; incubating the target nucleic acid, the probe, and the labeled competitor nucleic acid under conditions suitable for hybridization; determining the amount of labeled competitor nucleic acid bound to the nucleic acid probe; and quantifying the target nucleic acid based on the results the previous step.

### BRIEF DESCRIPTION OF THE DRAWINGS

FIG. 1 shows: A) Simulation of the binding kinetics of 1 nM competitor concentration in the presence of varying target concentration. B) Calibration curve. The symbols represent peak values of competitor curves.

FIG. 2 shows two-color real-time microarray imaging setup. Only a single color (green) is used in these experiments.

FIG. 3 shows: A) Experimental demonstration of CDDM with 10 nM competitor concentration. The symbols represent experimental data points while the solid curves are model fits (with R2 values greater than 0.93). Target concentrations are as indicated. B) Calibration curve. The symbols represent peak values of curve fits.

FIG. 4 shows CDDM with 1 nM competitor concentration, demonstrating detection of sub-nM target concentrations.

FIG. 5 shows homo-wt simulations using a 10 nM (wt) competitor and varying the concentration of wt target available to hybridize to the wt zone (A) and SNP zone (B).

FIG. 6 shows homogeneous mutated sample hybridization simulations using a competitor that is 10 nM, while varying the concentration of the mutated target available for hybridization to the wt zone (A) and the SNP zone (B).

FIG. 7 shows heterogeneous sample simulated using different concentrations of wt and SNP target while keeping the competitor at a concentration of 10 nM. A) hybridization curves of competitor binding to the wt zone when the concentration of the wt target is varied and the SNP target is constant at 2.5 nM, B) same description as A except binding to the SNP zone, C) hybridization curves of competitor binding to the wt zone when the concentration of the SNP target is varied and the wt is constant at 2.5 nM, D) same description as C except binding to the SNP zone.

FIG. 8 shows simulated hybridization curves for a multiplex hybridization with four components: target, competitor, and high and low background species. The composite curve (sum of all components) and effective background are also shown.

FIG. 9 shows hybridization curves representing the (A) target and (B) competitor kinetics as the number of background species increases from 0 to 5. Each progressive background species has a larger kd value to simulate decreasing stability. All species are present at 5-nM concentrations.

FIG. 10 shows hybridization curves representing the (A and C) target and (B and D) competitor kinetics as the concentration of a high (A and B) or low (C and D) affinity background is changed from 0 nM to 10 nM. For the red curves the TOI was 1 nM and for the green curves the TOI was 5 nM. The other four background species have a constant concentration of 1 nM and the competitor concentration is 5 nM.

FIG. 11 shows hybridization curves; simulations represented by the black line (taken from previous figures) and fits represented by the dashed lines, representing the (A) target and (B) competitor kinetics as the concentration of different background species concentrations are changed while the competitor concentration is constant at 5 nM. For the red curves the target of interest was 1.0 nM and for the green curves the target of interest was 5 nM.

FIG. 12 shows hybridization curves (experimental data represented by dots while fits are represented by solid lines) representing the competitor kinetics as the concentration of deletion (A and C, with fixed 1 nM TMM) and TMM (B and D, with fixed 1 nM deletion) are shifted from 0 nM to 10 nM while the competitor concentration is constant at 5 nM. In panels C and D, the red curves are for target concentration of 1.6 nM and the green curves are for target concentration 5 nM; target is absent in panels A and B.

### DETAILED DESCRIPTION OF THE INVENTION

A current research trend in molecular detection is the development of transduction mechanisms that do not require fluorescent labeling of the sample. Even though fluorescence-based detection is highly sensitive (to the level of a single molecule, Moerner 2003), it has the disadvantages of requiring extra steps in sample preparation, the labeling can influence the binding characteristics of the target molecule, and fluorophores are relatively costly. Label-free methods typically rely upon the shift in an electrical, mechanical, or optical resonance in proportion to the concentration of one or more species bound to a surface, which may result through the process of molecular capture. Traditional techniques include the quartz crystal micro balance (QCM) (Roederer 1983; Ngeh-Ngwainbi 1990) and surface plasmon resonance (SPR) (Liedberg 1983; Bianchi 1997). With SPR, for example, an optical resonance exists at a metal-dielectric interface which is sensitive to dielectric properties near the surface via the evanescent wave. These techniques (SPR in particular) are popular for protein binding studies, but are less effective for DNA due to the smaller molecular weight of sequence lengths in common use; sensitivities for short sequences (˜20-mers) are typically in the nM range (Wark 2005), but sub-nM sensitivities have been reported (Vaisocherova 2005). The sensitivity of these techniques also scale poorly when the size of the capture spot is reduced. As a result, numerous label-free transducers have been developed based upon localized surface plasmon (LSP) resonances in metal nanostructures (Okamoto 2000; Yonzon 2005; Rindzevicius 2005), optical resonance in dielectric resonators and microcavities (Boyd 2001; Altug 2005), and mechanical resonances in micro- and nano-cantilevers (Ilic 2005), for example. These methods promise to provide improved sensitivity and/or greater transducer density than QCM or SPR, but it is not clear that they will achieve the routine performance levels of fluorescence detection (with readout sensitivities in the 1-10 molecules/μm² range using commercial microarray substrates and scanners (Storhoff 2005). Further, work continues on new, more sensitive, methods of fluorescence-based detection.

### DEFINITIONS

By “competitor nucleic acid” is meant a nucleic acid that competes for binding to the probe with the target nucleic acid. The sequence of the competitor nucleic acid is generally known, although this is not necessary in some instances. The competitor nucleic acid can be a perfect match for the probe, or can have any given amount of sequence similarity to the probe.

By “binding pattern” is meant the way in which a nucleic acid hybridizes to the probe. This can be determined by observing the rate of binding of the nucleic acid to the probe as a function of time, such as in real time. The rate of binding is determined by the amount of individual nucleic acid molecules which have bound to the probe at a given point in time. Therefore, as the amount of individual nucleic acid molecules bound to the probe varies with time, so varies the binding pattern of the nucleic acid as a whole. For example, the total amount of target nucleic acid molecules bound to the probe at any given time can be measured, and the change in the number of these individual nucleic acid molecules bound to the probe over time can be measured, thereby determining the binding pattern of the target nucleic acid.

By “simultaneously” is meant, generally, at the same time. This can mean at the exact same moment, or within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 seconds or more of the same moment. One of skill in the art would be apprised of the meaning of “simultaneous.”

By “presence of target” is meant that a target nucleic acid is present in a given assay or sample. This can represent the presence of one or more individual target nucleic acid molecules.

By “displaced” is meant that a given nucleic acid molecule is no longer bound to the probe, and instead, another nucleic acid molecule of a different sequence has bound the probe. Such displacement can be temporary or permanent, and most commonly such displacement changes as a function of time, as given molecules bind and unbind the probe with some frequency. The nucleic acid which has displaced the original on the probe can have one or more nucleotides that differ from the original nucleic acid.

By “competitor” is meant a nucleic acid that competes with the target nucleic acid to bind the probe. Typically, the competitor nucleic acid shares some sequence similarity with the target, and can be identical in homology to the probe, or can vary by one or more nucleotides.

By “background nucleic acid” is meant nucleic acid that is neither the competitor nor the target. The sequence of such background can be known or unknown. The background nucleic acid can be labeled or unlabeled. Typically, the background nucleic acid is present in a sample as a contaminant, but it can also be put there on purpose to aid in detection. There can be a single species of nucleic acid present as the background, or there can be 2, 3, 4, 5, 6, 7, 8, 9, or 10 or more different nucleic acid sequences present in the background. The binding pattern of the background nucleic acid can be used to quantitate the target nucleic acid, for example.

### EMBODIMENTS

Disclosed herein is a method that takes advantage of fluorescence readout, but does not require the sample to be fluorescently labeled. This technique, termed Competitive Displacement Detection Method (CDDM), uses a known “competitor” molecular species that is fluorescently labeled and spiked into the sample. The binding kinetics of the competitor provides quantitative information about the unlabeled molecules of interest via the mechanism of competitive displacement (Zhang 2005; Bishop 2006; Bishop 2007).

In solution, real-time fluorescence readout can be obtained with FRET-based mechanisms (Henry 1999) to isolate binding events from background. At a surface, evanescent wave excitation can be used via a TIRF arrangement (Reichert 1989), a dielectric waveguide (Zhou 1991; Plowman 1996), or surface-enhanced fluorescence in a metallic film or nanostructure (Attridge 1991; Ditlbacher 2001; Malicka 2003; Liu 2003), for example. Nanoparticle scattering labels (Stimpson 1995; Taton 2000) could also be used on the competitor as an alternative to fluorescence. The technique for DNA detection using evanescent-wave excitation from a thick planar waveguide is demonstrated herein.

Disclosed herein are methods of determining sequence similarity of a target nucleic acid and a probe, the method comprising: bringing into contact the target nucleic acid, the probe, and a labeled competitor nucleic acid; simultaneously incubating the target nucleic acid, a probe, and the labeled competitor nucleic acid under conditions suitable for hybridization; determining the binding pattern of the competitor nucleic acid to the probe in the presence of the target nucleic acid; and determining sequence similarity of the target nucleic acid and the probe based on the results of the previous step.

Also disclosed herein are methods of detecting the presence of a target nucleic acid in a sample, the method comprising: simultaneously bringing into contact the sample, a probe, and a labeled competitor nucleic acid; incubating the sample, the probe, and the labeled competitor nucleic acid under conditions suitable for hybridization; determining the binding pattern of the competitor nucleic acid to the probe in the presence of the sample; and detecting the presence of the target nucleic acid based on the results of the previous step.

Disclosed herein is a method of quantifying a target nucleic acid, the method comprising: simultaneously bringing into contact a target nucleic acid, a probe, and a labeled competitor nucleic acid; incubating the target nucleic acid, the probe, and the labeled competitor nucleic acid under conditions suitable for hybridization; determining the amount of labeled competitor nucleic acid bound to the nucleic acid probe; and quantifying the target nucleic acid based on the results the previous step.

In the methods disclosed herein, the target nucleic acid can be labeled as well as the competitor nucleic acid. The target nucleic acid label can be distinct from the competitor nucleic acid label, so that they may be distinguished from each other. An example of the type of labeling includes fluorescent labels, which are described in more detail below. Furthermore, more than one labeled competitor nucleic acid can be used in the assay. For example, 2, 3, 4, 5, 6, 7, 8, 9, 10, 11, 12, 13, 14, 15, or more different competitor nucleic acids can be used. In one example, each labeled competitor nucleic acid is labeled with a different label.

A wide range of detection methods is applicable to the methods of the invention. As desired, detection may be either quantitative or qualitative. The invention array can be interfaced with optical detection methods such as absorption in the visible or infrared range, chemiluminescence, and fluorescence (including lifetime, polarization, fluorescence correlation spectroscopy (FCS), and fluorescence-resonance energy transfer (FRET)). Furthermore, other modes of detection such as those based on optical waveguides PCT Publication (WO 96/26432 and U.S. Pat. No. 5,677,196), surface plasmon resonance, surface charge sensors, and surface force sensors are compatible with many embodiments of the invention. Evanescent wave excitation can be accomplished via an optical waveguide, for example, which can be substantially planar, and can comprise thin dielectric or metallic films. Alternatively, technologies such as those based on Brewster Angle microscopy (BAM) (Schaaf et al., Langmuir, 3:1131-1135 (1987)) and ellipsometry (U.S. Pat. Nos. 5,141,311 and 5,116,121; Kim, Macromolecules, 22:2682-2685 (1984)) could be applied. Quartz crystal microbalances and desorption processes (see for example, U.S. Pat. No. 5,719,060) provide still other alternative detection means suitable for at least some embodiments of the invention array. An example of an optical biosensor system compatible both with some arrays of the present invention and a variety of non-label detection principles including surface plasmon resonance, total internal reflection fluorescence (TIRF), Brewster Angle microscopy, optical waveguide lightmode spectroscopy (OWLS), surface charge measurements, and ellipsometry can be found in U.S. Pat. No. 5,313,264. Furthermore, nanoparticle scattering labels, or Raman scattering labels, can also be used with the methods disclosed herein.

Regarding the concentrations of the competitor nucleic acid versus the target nucleic acid, the proportions can vary with respect to each other. One of skill in the art can determine what the concentration of each should be, depending upon the similarity of the nucleic acids to each other, and to the probe nucleic acid. The competitor nucleic acid can be in higher concentrations, such as 2, 3, 4, 5, 6, 7, 8, 9, 10, or more times greater, or any fraction in between, compared to the target nucleic acid. Alternatively, the target nucleic acid can be present in a higher concentration than the competitor nucleic acid. It can be present at 2, 3, 4, 5, 6, 7, 8, 9, 10, or more times greater concentration, or any fraction in between, compared to the competitor nucleic acid. Alternatively, the competitor nucleic acid and target nucleic acid can be present in the same amount.

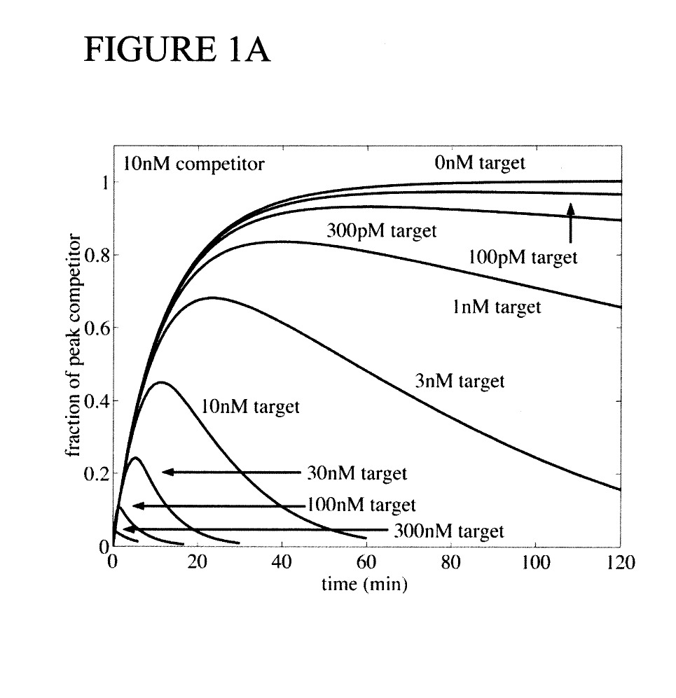

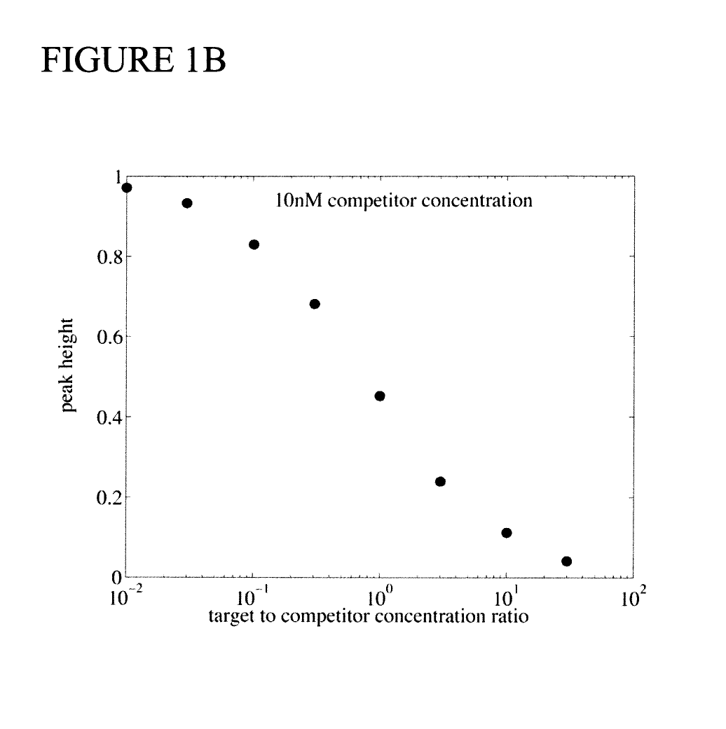

As described herein, the hybridization pattern can differ as a function of time. For example, the labeled competitor nucleic acid can initially dominate hybridization, and then be displaced by the target nucleic acid. An example of such a pattern can be found in FIG. 1.

The target nucleic acid and the probe are complementary in sequence. For example, they can be 100% complementary to each other. Alternatively, they can vary in complementation, which will be reflected in the binding stringency of the target for the probe. Furthermore, the competitor nucleic acid and the probe can be 100% complementary, or can vary as well. When the nucleic acids vary, it is defined as a difference in hybridization. Hybridization can also vary as a function of other parameters, which are disclosed below.

The term hybridization typically means a sequence driven interaction between at least two nucleic acid molecules, such as a probe and a competitor or target nucleic acid. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is not only affected by sequence similarity, but is also affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

The methods disclosed herein can be used with a solid support, such as a microarray. More than one probe nucleic acid can be used, and different types of probes can be used within the same assay.

The target nucleic acid sequences, competitor nucleic acids, and probes can be coupled to a substrate. Doing so is useful for a variety of purposes including immobilization of the reaction or reaction products, allowing easy washing of reagents and reactions during an assay, aiding identification or detection of structured probes, and making it easier to assay multiple samples simultaneously. In particular, immobilization of target sequences allows the location of the target sequences in a sample or array to be determined. For example, a cell or chromosome spread can be probed in the disclosed method to determine the presence and location of specific target sequences within a cell, genome, or chromosome.

Solid-state substrates to which target samples, target sequences, or structured probes can be attached can include any solid material to which nucleic acids can be attached, adhered, or coupled, either directly or indirectly. This includes materials such as acrylamide, cellulose, nitrocellulose, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. The solid support can also be made of semiconductor materials such as silicon, GaAs, quartz, silicon nitride, silicon oxynitride, metal oxides (e.g. TiO₂, Al₂O₃, Ta₂O₅). The solid support can be porous or non-porous. Solid-state substrates can have any useful form including thin films or membranes, beads, bottles, dishes, fibers, woven fibers, shaped polymers, particles and microparticles. Preferred forms for solid-state substrates are flat surfaces, especially those used for cell and chromosome spreads.

When structured probes are immobilized, it is preferred that the first ends of the probes are coupled to the solid support. The solid support can be made up of a plurality of probes located in a plurality of different predefined regions of the solid support. Preferably, the probes collectively correspond to a plurality of target nucleic acid sequences.

The solid support can be made up of at least one thin film, membrane, bottle, dish, fiber, woven fiber, shaped polymer, particle, bead, or microparticle, or at least two thin films, membranes, bottles, dishes, fibers, woven fibers, shaped polymers, particles, beads, microparticles, or a combination.

Methods for immobilization of nucleic acids to solid-state substrates are well established. In general, target samples and target sequences can be immobilized on a substrate as part of a nucleic acid sample or other sample containing target sequences. Target sequences and structured probes can be coupled to substrates using established coupling methods. For example, suitable attachment methods are described by Pease et al., Proc. Natl. Acad. Sci. USA 91(11):5022-5026 (1994), Guo et al., Nucleic Acids Res. 22:5456-5465 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995).

Methods for producing arrays of nucleic acids on solid-state substrates are also known. Examples of such techniques are described in U.S. Pat. No. 5,871,928 to Fodor et al., U.S. Pat. No. 5,54,413, U.S. Pat. No. 5,429,807, and U.S. Pat. No. 5,599,695 to Pease et al. Microarrays of RNA targets can be fabricated, for example, using the method described by Schena et al., Science 270:487-470 (1995).

Although preferred, it is not required that a given array of target samples, target sequences, or structured probes be a single unit or structure. The set of sequences or probes may be distributed over any number of solid supports. For example, at one extreme, each target sequence, each target sample, or each structured probe may be immobilized in or on a separate surface, reaction tube, container, or bead.

A variety of cell and nucleic acid sample preparation techniques are known and can be used to prepare samples for use in the disclosed method. For example, metaphase chromosomes and interphase nuclei can be prepared as described by Cremer et al., Hum Genet. 80(3):235-46 (1988), and Haaf and Ward, Hum Mol Genet. 3(4):629-33 (1994), genomic DNA fibers can be prepared as described by Yunis et al., Chromosoma 67(4):293-307 (1978), and Parra and Windle, Nature Genet. 5:17-21 (1993), and Halo preparations can be prepared as described by Vogelstein et al., Cell 22(1 Pt 1):79-85 (1980), and Wiegant et al., Hum Mol. Genet. 1(8):587-91 (1992).

Also disclosed herein is that the target nucleic acid can comprise a single nucleotide polymorphism (SNP). The methods disclosed herein are particularly useful with SNPs. Critical advantages of these methods as applied to SNPs are that non-specific background fluorescence in minimized, because the samples are not fluorescently labeled, resulting in increased assay sensitivity, and analysis is based on binding kinetics. The latter allows for one to obtain more reliable data over shorter times compared to the “quasi-equilibrium” assumption of current microarray analysis. This approach can be readily scaled for parallel interrogation of multiple SNPs on mtDNA and relevant SNPs on genomic DNA for other diseases. CDDM can also be used to utilize thin-film planar waveguides (Herron 2003) (rather than microscope slides), which can provide the additional improvement in sensitivity (˜100 times) to bypass DNA amplification and apply denatured mtDNA directly to the sensing array, providing “sample to answer” capability.

The nucleic acids disclosed herein can be selected from the group consisting of DNA, RNA, or a combination thereof. There are multiple examples of types of DNA and RNA known in the art. Examples include, but are not limited to, cDNA, mtDNA, mRNA, miRNA, and siRNA. The nucleic acids disclosed herein can also be peptide nucleic acids.

The probes, competitors, and targets can be a nucleic acid, and can also can be, or include regions of, peptide nucleic acids and other oligonucleotide analogues. The structured probes also can include nucleoside and nucleotide analogues. In particular, the target probe portion, the complementary portions, and the detection portion can be chimeric; containing any combination of standard nucleotides, nucleotide analogues, nucleoside analogues, and oligonucleotide analogues.

As used herein, oligomer refers to oligomeric molecules composed of subunits where the subunits can be of the same class (such as nucleotides) or a mixture of classes (such as nucleotides and ethylene glycol). It is preferred that the disclosed probes be oligomeric sequences, non-nucleotide linkers, or a combination of oligomeric sequences and non-nucleotide linkers. It is more preferred that the disclosed probes be oligomeric sequences. Oligomeric sequences are oligomeric molecules where each of the subunits includes a nucleobase (that is, the base portion of a nucleotide or nucleotide analogue) which can interact with other oligomeric sequences in a base-specific manner. The hybridization of nucleic acid strands is a preferred example of such base-specific interactions. Oligomeric sequences preferably are comprised of nucleotides, nucleotide analogues, or both, or are oligonucleotide analogues.

A non-nucleotide linker can be any molecule that can be covalently coupled to an oligomeric sequence. Preferred non-nucleotide linkers are oligomeric molecules formed of non-nucleotide subunits. Examples of such non-nucleotide linkers are described by Letsinger and Wu, (J. Am. Chem. Soc. 117:7323-7328 (1995)), Benseler et al., (J. Am. Chem. Soc. 115:8483-8484 (1993)) and Fu et al., (J. Am. Chem. Soc. 116:4591-4598 (1994)). Preferred non-nucleotide linkers, or subunits for non-nucleotide linkers, include substituted or unsubstituted C₁-C₁₈ straight chain or branched alkyl, substituted or unsubstituted C₂-C₁₈ straight chain or branched alkenyl, substituted or unsubstituted C₂-C₁₈ straight chain or branched alkynyl, substituted or unsubstituted C₁-C₁₈ straight chain or branched alkoxy, substituted or unsubstituted C₂-C₁₈ straight chain or branched alkenyloxy, and substituted or unsubstituted C₂-C₁₈ straight chain or branched alkynyloxy. The substituents for these preferred non-nucleotide linkers (or subunits) can be halogen, cyano, amino, carboxy, ester, ether, carboxamide, hydroxy, or mercapto.

As used herein, nucleoside refers to adenosine, guanosine, cytidine, uridine, 2′-deoxyadenosine, 2′-deoxyguanosine, 2′-deoxycytidine, or thymidine. A nucleoside analogue is a chemically modified form of nucleoside containing a chemical modification at any position on the base or sugar portion of the nucleoside. As used herein, nucleotide refers to a phosphate derivative of nucleosides as described above, and a nucleotide analogue is a phosphate derivative of nucleoside analogues as described above. The subunits of oligonucleotide analogues, such as peptide nucleic acids, are also considered to be nucleotide analogues.

As used herein, oligonucleotide analogues are polymers of nucleic acid-like material with nucleic acid-like properties, such as sequence dependent hybridization, that contain at one or more positions a modification away from a standard RNA or DNA nucleotide. A preferred example of an oligonucleotide analogue is peptide nucleic acid. The internucleosidic linkage between two nucleosides can be achieved by phosphodiester bonds or by modified phospho bonds such as by phosphorothioate groups or other bonds such as, for example, those described in U.S. Pat. No. 5,334,711.

A useful and accessible class of nucleic acid analogs is the family of peptide nucleic acids (PNA) in which the sugar/phosphate backbone of DNA or RNA has been replaced with acyclic, achiral, and neutral polyamide linkages. The 2-aminoethylglycine polyamide linkage in particular has been well-studied and shown to impart exceptional hybridization specificity and affinity when nucleobases are attached to the linkage through an amide bond.

Aminoethylglycine PNA oligomers typically have greater affinity, i.e. hybridization strength and duplex stability for their complementary PNA, DNA and RNA, as exemplified by higher thermal melting values (Tm), than the corresponding DNA sequences. The melting temperatures of PNA/DNA and PNA/RNA hybrids are much higher than corresponding DNA/DNA or DNA/RNA duplexes (generally 1° C. per bp) due to a lack of electrostatic repulsion in the PNA-containing duplexes. Also, unlike DNA/DNA duplexes, the Tm of PNA/DNA duplexes are largely independent of salt concentration. The 2aminoethylglycine PNA oligomers also demonstrate a high degree of base-discrimination (specificity) in pairing with their complementary strand. Specificity of hybridization can be measured by comparing Tm values of duplexes having perfect Watson/Crick complementarity and those with one or more mismatches. The degree of destabilization of mismatches, measured by the decrease in Tm (ΔTm), is a measure of specificity. In addition to mismatches, specificity and affinity are affected by structural modifications, hybridization conditions, and other experimental parameters. The neutral backbone of PNA also increases the rate of hybridization significantly in assays where either the target, template, or the PNA probe is immobilized on a solid substrate. Without any electrostatic repulsion, the rate of hybridization is often much higher for PNA probes than for DNA or RNA probes in applications such as Southern blotting, northern blots, or in situ hybridization experiments. Unlike DNA, PNA can displace one strand, “strand invasion”, of a DNA/DNA duplex. With certain DNA sequences, a second PNA can further bind to form an unusually stable triple helix structure (PNA).sub.2/DNA. PNA have been investigated as potential antisense agents, based on their sequence-specific inhibition of transcription and translation. PNA oligomers themselves are not substrates for polymerase as primers or templates, and do not conduct primer extension with nucleotides.

The target nucleic acid can be amplified before being put in contact with the probe and the labeled competitor nucleic acid. However, as the assay disclosed herein is very sensitive, the target nucleic acid need not be amplified before being used in the assay disclosed herein. When the target nucleic acid is amplified, polymerase chain reactions (PCR) can be used (Mullis, K., U.S. Pat. No. 4,683,202; Saiki, R. K., et al., Enzymatic Amplification of .beta.-Globin Genomic Sequences and Restriction Site Analysis for Diagnosis of Sickle Cell Anemia, In PCR: A practical approach, M. J. McPherson, P. Quirke, and G. R. Taylor, Eds., Oxford University Press, 1991).

In this aspect of the present invention, the detection method involves PCR amplification of nucleotide sequences within the target nucleic acid. In this aspect, a target nucleic acid, which may be immobilized, is contacted with a plurality of, two of which hybridize to complementary strands, and at opposite ends, of a nucleotide sequence within the target nucleic acid. Repeated cycles of extension of the hybridized sequence-specific oligonucleotides, optionally by a thermo-tolerant polymerase, thermal denaturation and dissociation of the extended product, and annealing, provide a geometric expansion of the region bracketed by the two probes. The product of such a polymerase chain reaction therefore is a double-stranded molecule consisting of two strands, each of which comprises a sequence-specific probe. In this aspect of the present invention, at least one of the sequence-specific oligonucleotides is a sequence-specific probe such that the double stranded polymerase chain reaction product has a distinctive ratio of charge to translational frictional drag.

In yet another aspect, the polymerase chain reaction product formed is analyzed under denaturing conditions, providing separated single stranded products. In this aspect, at least one of the single stranded products comprises both a label and a sequence-specific primer such that the single-stranded product derived from double stranded polymerase chain reaction product has a distinctive ratio of charge to translational frictional drag. As is well known in the art, such a single-stranded product may also be generated by carrying out the PCR reaction with limiting amounts of one of the two sequence-specific probes used as a primer. By using distinctive sequence-specific nucleic acids or probes as primers, the PCR reaction can detect many selected regions within one or more target polynucleotides in a single assay by allowing separation of one PCR product from another. Moreover, those skilled in the art will recognize that using various combinations of primers provides additional ways to generate distinctive PCR products. For example, a combination of a probe and a second primer pair in the PCR reaction generates a PCR product with a single strand. On the other hand, a combination of a probe and a second probe, which is also mobility-modified, generates a PCR product having both strands that are mobility-modified, thus distinguishing itself from the PCR product with one strand. Thus, by varying the type of mobility-modifying group and the nucleic acid strands that are mobility-modified, the embodiments enlarge the capacity to detect multiple target segments.

The effects of binding of additional non-target nucleic acid sequences on the binding pattern of the competitor nucleic acid sequences can be used to determine the sequence similarity of the target nucleic acid and the probe. Curve fitting of the binding patterns of the competitor nucleic acid and target nucleic acid sequences can also be used to determine the sequence of the target nucleic acid.

Curve fitting can allow for the determination of the sequence of the target nucleic acid. In one example, curve fitting of the binding patterns of the labeled nucleic acid to two or more probe sequences can be used to determine sequence similarity between the target nucleic acid and the probe. For example, 2, 3, 4, 5, 6, 7, 8, 9, 10, 11, 12, 13, 14, 15, or more probe sequences can be used to determine the sequence of the target nucleic acid. Curve fitting of the binding patterns of multiple competitor nucleic acid sequences can used determine the sequence similarity of the target nucleic acid and the probe. Furthermore, curve fitting of the binding patterns of multiple competitor nucleic acid sequences can be used to determine the sequence similarity of two or more target nucleic acid sequences and their corresponding probes.

Probes and competitor nucleic acids, and any other oligonucleotides, can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol. 65:610-620 (1980), (phosphotriester method). Protein nucleic acid molecules can be made using known methods such as those described by Nielsen et al., Bioconjug. Chem. 5:3-7 (1994).

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

The following examples are put forth to provide those of ordinary skill in the art with a complete disclosure and description of how the compositions and/or methods claimed herein are made and evaluated, and are intended to be purely exemplary of the invention and are not intended to limit the scope of what the inventors regard as the invention. Efforts have been made to ensure accuracy with respect to numbers (e.g., amounts, temperature, etc.), but some errors and deviations should be accounted for. The present invention is more particularly described in the following examples which are intended as illustrative only because numerous modifications and variations therein will be apparent to those skilled in the art.

### K. EXAMPLES

### Example 1

### A Sensitive Method for Label-Free Molecular Detection

Competitive Displacement Mechanism

In order to understand the mechanism of competitive displacement, consider the chemical reaction of two different species (target and competitor) to a single probe species:

dB/dt=kaC(Rt−B−Bcomp)−kdB (1)

dBcomp/dt=ka,compCcomp(Rt−B−Bcomp)−kd,compBcomp (2)

where B represents bound concentrations, ka association rate constants, C target solution concentrations, Rt concentration of probe molecules, and kd dissociation rate constants. In the case of surface capture, mass transport from solution to the surface can be described by Fick's Law:

d(C+Ccomp)/dt=D∇2(C+Ccomp) (3)

where D is the diffusion coefficient, assumed the same for the two species, and convective transport is neglected. Under the equilibrium condition (i.e. all time derivatives are zero), the occupation fraction of target and competitor to the probes is

B/Rt=Ψ=kaC/[(kaC+ka,compCcompkd)/(kd,comp+kd)] (4)

Bcomp/Rt=Ψcomp=(ka,compCcomp)/[ka,compCcomp+(kaCkd,comp)/(kd+kd,comp)] (5)

assuming that solution concentrations are not significantly depleted by binding to the probes. Because association rates are always connected with their respective solution concentrations, the dynamic range of selectivity (i.e. the ability to discriminate one species from the other) is determined by the dissociation rates (Bishop 2006).

Of a less general, but highly illustrative, nature, Eqs. 1 and 2 can be solved, assuming C

constant over all time (the so-called well-mixed case):

B(t)/Rt=Y−ae−t1t−be−t2t (6)

Bcomp(t)/Rt=Ycomp+ge−t1t−de−t2t (7)

where the a, b, g, and d coefficients originate from the rate constants and concentrations of the target and competitor species, and a+b=Y and d−g=Ycomp. For the target, equation 6, the exponential terms add to produce a monotonically-increasing curve; however, for the competitor, equation 7, non-monotonic behavior is possible. At early times, the t2 term controls a monotonic increase, and, during displacement (i.e. t>>t2), the t1 term controls with exponential decrease. Although not shown explicitly from Eqs. 6 and 7, the kinetics of the competitor strongly depend on the concentration of the target species, and hence forms the basis of CDDM. For the simulations and experiments, we have chosen a simple model system consisting of 20-mer sequences: target CGAGGGCAGCAATAGTACAC (SEQ ID NO: 1), competitor CGAGGGCAGCATTAGTACAC (SEQ ID NO: 2) which differs from the target by a single base, and probe that is a perfect complement to the target. During simulations association rates ka=ka,comp=10⁶ M−1 s−1, dissociation rates kd=3.4×10−6 s−1 and kd,comp=3.7×10−3 s−1, a diffusion coefficient D=1.3×10−10 m2 s−1, and probe concentration Rt=10-11 M·m were assumed. These parameters produce results which are in reasonable agreement with the experimental results in the following section. Further computational details can be found in Bishop 2006.

FIG. 1A shows results from simulations of the binding kinetics of the competitor (with 10 nM solution concentration C_{comp}) as a function of the solution concentration of the target species. One distinguishable feature from these curves is the maximum height of B_{comp}, which depends on C (which represents the unknown/unlabeled target concentration). Using the maximum height of B_{comp}, a calibration curve can be generated, as shown in FIG. 1B. Note that at very large or very small target to competitor concentration ratios, the peak height (normalized to the equilibrium value in the absence of the target) asymptotically approaches its limiting values of 0 and 1, respectively. Combined with the SNR in detection (which determines the smallest changes that can be measured), this effect ultimately limits the dynamic range of C. The dynamic range can be extended towards lower C by measuring the exponential decay during the displacement regime, but this requires longer acquisition times approaching equilibrium.

Further, dynamic range can be shifted in concentration space by either changing C_{comp} or by altering the ratio of kd values by changing the competitor sequence or the temperature of the reaction. Another approach to lower the detection limit is to reduce the value of Rt so that competition can occur with low target concentration (Bishop 2006).

Label-Free Detection Experiments

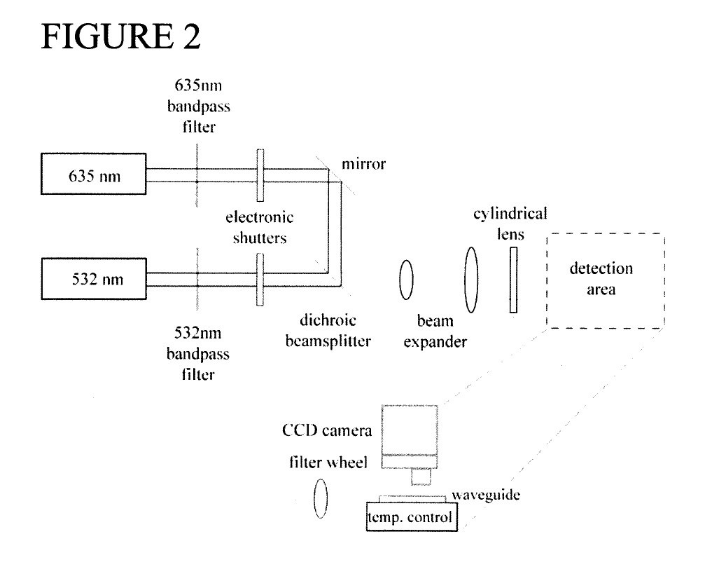

Experimental evidence of competitive displacement was provided in Bishop 2007. Here, the change in competitor kinetics as a function of target concentration is demonstrated using the same sequences as used during modeling. The experimental setup used to perform CDDM is illustrated in FIG. 2. A 532 nm laser was end-fire coupled into a quartz microscope slide which acted as an optical waveguide. Real-time detection of hybridization by a CCD camera (Santa Barbara Instruments ST-7XMEI) was facilitated by the evanescent field due to total internal reflection inside the waveguide, with the detected fluorescence signal proportional to Bcomp(t). The imaged area encompassed reference and hybridization spots, where reference spots consisted of a fluorescently-labeled probe sequence that was non-interacting with the other sequences in the experiment. The camera was cooled to −5° C. using internal Peltier devices and by water from a refrigeration unit. Each frame captured by the camera was exposed for 2.5 seconds and then saved for post processing. A TTL signal from the camera shutter was used to modulate the laser output in order to reduce photobleaching during the time interval between acquisitions. Further experimental details, such as surface modification chemistry, target and probe preparation, and probe immobilization can be found in (Bishop 2007).

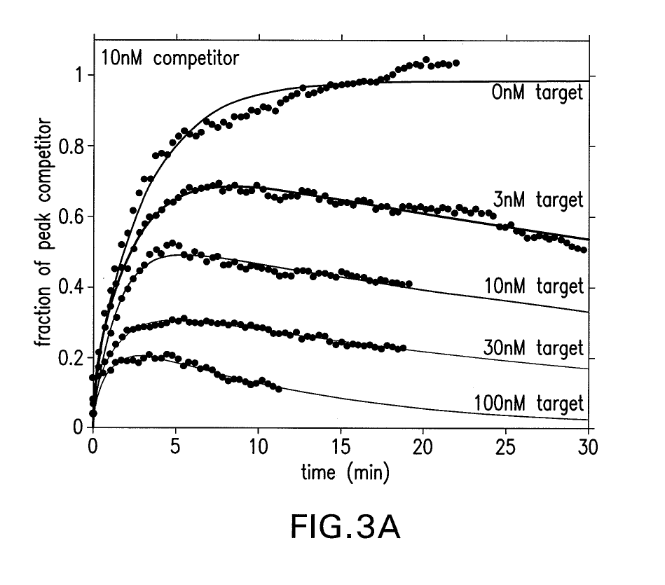

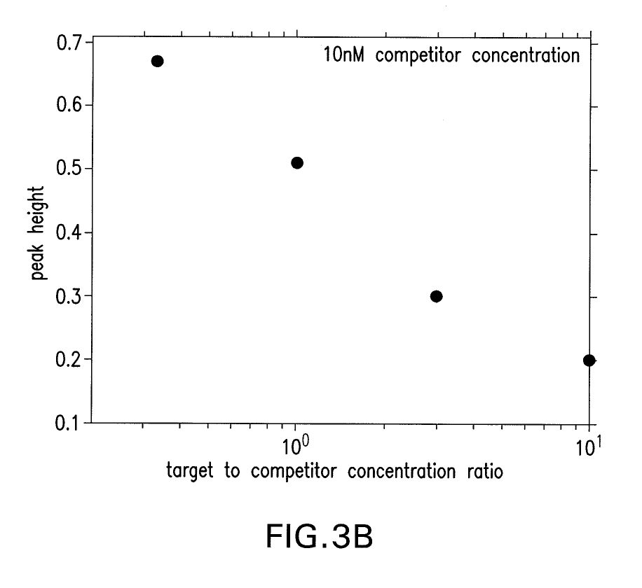

The first set of experimental results were obtained with a relatively high competitor concentration of 10 nM in order to reduce the time per hybridization run, as shown in FIG. 3A. Target concentrations were varied from 0 nM to 100 nM; in each experiment, 250 mL of solution containing target and competitor was dispensed over the hybridization spots. Before each hybridization experiment, the duplexes formed during the previous experiment were melted by first raising the surface temperature above the melting temperature and then washing with buffer. Even though this technique improves the repeatability of the results, only a limited number of regeneration cycles can be employed before degradation of the probes occurs. Very good qualitative agreement with simulations is obtained. Further, since the concentrations used were relatively high, Eqs. 6 and 7 can be used to fit the experimental data, as indicated by the curves in FIG. 3A. Using these fitted curves, a calibration curve can be generated which relates the maximum height of the competitor curve to the concentration of the target, as shown in FIG. 3B. One important aspect to note is that the calibration used here does not require equilibrium be reached; all that is necessary is that the displacement regime be reached so that the peak height can be determined.

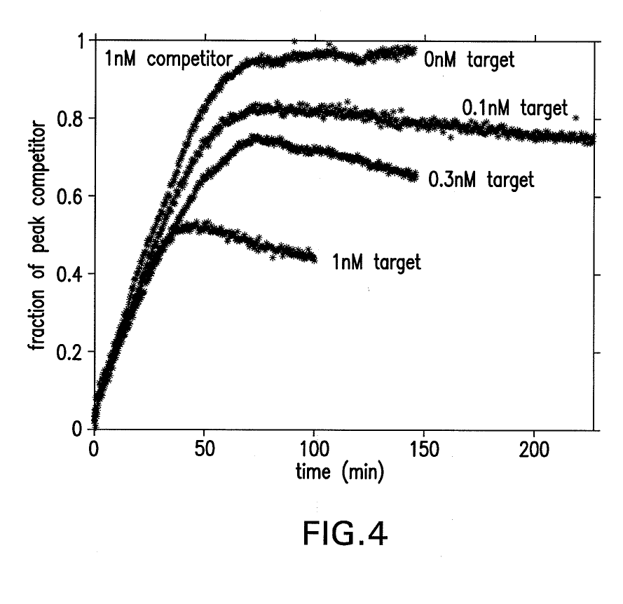

As the concentration of target decreases, the time needed to reach the displacement regime increases. Further, at low concentrations with respect to the competitor, the ability to discriminate via the height of the competitor curve diminishes (see FIG. 1B); in these cases, the exponential decay can be used, but this requires longer acquisition times, as stated previously. Even though microarray hybridizations are typically performed for 16 to 18 hours to try to reach equilibrium with lower concentrations, CDDM can be performed at these lower concentrations over shorter times by reducing the competitor concentration, thus shifting the dynamic range. FIG. 4 shows experimental curves with 1 nM competitor concentration and demonstrates sub-nM detection of the target. These reactions are more strongly diffusion controlled, so Eqs. 6 and 7 are less applicable in fitting. Nevertheless, it is clear from the experimental curves that concentrations below 0.1 μM should be detectable with this system.

Discussion and Conclusion

A new label-free detection method, CDDM, has been demonstrated, in which competitive displacement is used to indirectly detect a primary target. The preliminary experimental results show good correlation with simulation results and theory. Using CDDM it was shown experimentally that a sensitivity of one-tenth the competitor concentration with a dynamic range of detection greater than two orders of magnitude is achievable. Another aspect of sensitivity lies with the transduction mechanisms. In these experiments, a thick-film (˜1 mm) planar waveguide was used to excite fluorescence. Utilizing a thin-film waveguide (Plowman 1996; Attridge 1991) (1 mm, for example) reduces the effective mode size by about a factor of 1000, thereby increasing the intensity at the surface by roughly the same factor. In the shotnoise limit of detection, this results in an improvement in SNR by the square-root of the intensity enhancement factor (Blair 2001), or by about 30 in this case. More advanced approaches promise to improve the detection limits even further (Ditlbacher 2001; Malicka 2003; Liu 2004; Blair 2001). It is conceivable then that sufficient sensitivity in fluorescence-based detection can be obtained to render molecular amplification steps (such as cell culture or PCR) unnecessary in some circumstances; the additional advantage offered by CDDM is that sample preperation can be further simplified by not requiring a labeling step. In the case of DNA, the necessary sample preparation steps consist of cellular disruption, DNA isolation, and shearing to a desired average length. Aside from sensitivity limitations, the quantitative capability of CDDM depends upon a method of calibration of the fluorescence signals. A simple calibration method is to use calibration spots, where probe and target sequences associated with those spots are designed to be non-interacting with other sequences in the system, but with similar equilibrium binding constants as the actual competitor molecules. This has the advantage of allowing calibration and experiment to be performed simultaneously. Another method is to simply run an experiment with the competitors only so that their native kinetics can be determined, then reuse the array (by melting of surface bound duplexes, for example) with both competitor and sample.

### Example 2

### Competitive Displacement Detection Method

Disclosed herein is a method to perform real-time SNP microarray analysis by monitoring non-linear kinetic behavior of known competitors in the presence of unlabeled targets (CDDM). Critical advantages of this method are that non-specific background fluorescence in minimized, because the samples are not fluorescently labeled, resulting in increased assay sensitivity, and analysis is based on binding kinetics. The latter allows for one to obtain more reliable data over shorter times compared to the “quasi-equilibrium” assumption of current microarray analysis. CDDM is a straightforward assay, which requires only moderate instrumental investments and low cost consumables. This approach can be readily scaled for parallel interrogation of multiple SNPs on mtDNA and relevant SNPs on genomic DNA for other diseases. CDDM can also be used to utilize thin-film planar waveguides (Herron 2003) (rather than microscope slides), which can provide the additional improvement in sensitivity (˜100 times) to bypass DNA amplification and apply denatured mtDNA directly to the sensing array, providing “sample to answer” capability.

The method disclosed herein was demonstrated to produce quantitative data for an A-T mutation within the context of a simple 20mer model system using a microarray surface hybridization format without auxiliary enzymatic steps or sample labeling (Bishop 2007) The purpose is to validate the robustness of CDDM in the more complex environment of clinical DNA samples (i.e. on longer PCR products with the background of genomic DNA, primers, nucleotides, DNA polymerases and PCR buffer) and to develop diagnostic CDDM heteroplasmy assays.

CDDM heteroplasmy analysis is characterized using one SNP and a 4 zone array, with respect to the dynamic range of CDDM and reliability of results in a complex environment. Once the dynamic range of heteroplasmy quantification is optimized to the required value of >20 (i.e. sensitivity of better than 5% of relative SNP content) in the presence of PCR background, CDDM is further validated by parallel heteroplasmy analysis of 13 SNP loci using a 70 zone array (28 SNP probes and 26 wt probes in duplicates, positive and negative control zones for background subtraction and signal normalization). Lastly, a comparative study of results using CDDM and the single nucleotide extension method on sequence verified patient samples is performed.

Characterization of CDDM for Heteroplasmy Analysis on G11778A Mutation Locus.

The sensitivity limits and dynamic range of CDDM is characterized using a single high frequency pathological mutation, G11788A in the ND4 gene of mtDNA. A pair of primers encompassing this locus are used. Short asymmetric PCR products are obtained by amplifying the sequence between positions 11755 and 11866 (115 bp) using total DNA preparations from blood samples. PCR reactions, without further purification, are mixed with a calibrated solution of fluorescently labeled competitor target—a synthetic oligonucleotide sequence (Alexa488, 60mer) complementary to the wild type probe, which is the basis of CDDM. Two sensing zones (in duplicate) on the surface are functionalized using wt (G11778) and SNP (A11778) 60mer synthetic oligonucleotides. Accordingly, the labeled competitor forms a perfectly matched hybrid on the wt zone and a mismatched (A-C) hybrid on the SNP zone. Real-time hybridization of the competitor to the sensing zones is monitored using a custom experimental setup. The concentrations of the competitor are optimized with respect to standard PCR product concentrations. By utilizing sequence verified DNA samples accuracy and dynamic range are determined, i.e. relative amounts of heteroplasmy, which are detectable using CDDM. BVetter than 5% heteroplasmy sensitivity with 90% accuracy is achieved.

Scaling Up CDDM to Interrogate Multiple Mutations.

CDDM can, in parallel, interrogate 13 loci of mtDNA, which harbor pathological mutations. The same approach described above is used, however now 13 different amplicons are mixed with 13 different labeled competitors and applied as a multi-component sample to the sensing array. Each amplicon is interrogated in its addressable spots, and results are assessed for accuracy using sequence verified DNA samples. Design of the probes and competitors re performed using Visual OMP (DNA Software) and UPG (Portland Bioscience), with emphasis on minimizing intramolecular folding and cross hybridization of competitors in solution. One locus is of particular interest, since it harbors two different mutations in the same position: T8993C and T8993G in the ATP6 gene. Differences in the kinetic behaviors of the competitors on the zones matched to each of the SNPs are interrogated, which can lead to a reduced number of sensing zones. Another interesting locus encompasses A8344G and T8356C. In this case, a single wt competitor is used to interrogate spatially distinct mutation spots. Competitor design in this case is targeting maximal thermodynamic discrimination between wt and two proximal mutations.

Competitive Displacement Detection Method (CDDM)

In order to study multi-target capture on the surface of a microarray, a mass transport/kinetic model was developed (Bishop 2006; Bishop 2007). In the presence of a mixed sample with sequences complementary (i.e. the ‘match’) and non-complementary (i.e. ‘mismatches’) to the capture probe, mismatched species bind to the sensing zone in early hybridization times, and can dominate the early growth if sufficiently complementary to the probe and present at higher concentrations than the match, but are eventually displaced from the sensing zone by the match as the surface reaction progresses to the equilibrium state. The term “competitive displacement” was coined to describe this process. These theoretical results have been confirmed experimentally using a model system based on synthetic 20mer wt and SNP oligonucleotides (Bishop 2007). Practically important implications of this model are that a) there are multiple species whose sequences confer different affinities which concurrently bind to each sensing zone, so that interpretation of end point fluorescence scanning is problematic, especially in the presence of several similar species (wt and SNPs) and when equilibrium is not attained, and b) the kinetics of a mismatched sequence (i.e. initial growth followed by displacement) is a function of relative affinity and relative concentration of the match.

These considerations prompted the development of a novel tagless DNA quantitative assay, where the concentration of unlabeled targets is estimated indirectly by the kinetic behavior of spiked-in labeled mismatched competitor sequences. FIG. 1A demonstrates results of simulations for 20-mer sequences, with the target and competitor differing by a single base and the probe a perfect complement to the target. Kinetic rate constants were estimated using a thermodynamic model (Bishop 2006) based upon entropy and enthalpy values obtained from MeltCalc. Titration of the unlabeled target was performed in the presence of the fixed concentration of the labeled competitor. As a first approximation the maximum value of the competitor signal was used as a measure of the target concentration. FIG. 1B shows the resulting calibration curve, where the competitor signal maxima are plotted as a function of the target concentration. The linear portion of this curve represents the concentration range of quantification, which can be shifted depending on the concentration of the competitor used.

Real-Time Microarray Platform and CDDM Experiments

In order to perform kinetic analysis of microarray experiments, a novel and flexible real-time microarray platform, as illustrated in FIG. 3. Dual color excitation of fluorescence proximal to the surface is achieved by end-firing laser beams into a planar waveguide (Herron 2003) in this case, a quartz microscope slide (Taitt 2005; Bishop 2007). The condition of total internal reflection (TIR) ensures that only surface bound labeled species are exposed, via the evanescent field, while bulk solution excitation is negligible. The surface of the planar waveguide is functionalized by short single stranded oligonucleotides complementary to the target loci in predefined (addressable) positions. The hybridization chamber was created using an optically transparent cover (MAUI, BioMicro Systems), and the cover and waveguide substrate sit atop a Peltier device which allows temperature control. During hybridization, real-time capture of fluorescence signals from the surface is accomplished with a digital CCD camera (Santa Barbara Instruments) placed above the hybridization chamber. The camera is equipped with a computer controlled filter wheel to facilitate multi-color imaging. With CDDM, only a single color channel is needed. The images are collected frame by frame and analyzed using a MATLAB package.

Experimental demonstration of CDDM has been performed using 20mer sequences—cgagggcagcaatagtacac (target) (SEQ ID NO: 1) and cgagggcagcaTtagtacac (Cy-3 labeled competitor), SEQ ID NO: 2 and a probe sequence complementary to the target. FIG. 3A shows the experimental curves obtained by monitoring the competitor in the presence of different target concentrations. Under a simple approximation that neglects mass transport (the so-called “well-mixed” condition), the kinetics of the competitor can be described mathematically by the bi-exponential function

B_{comp}(t)=Ψ_{comp}+γe^{-τ}^{₁}^{t}−δe^{-τ}^{₂}^{t} (1)

where B_{comp} is the measured signal, Ψ_{comp} is the equilibrium value, γ−δ=Ψ_{comp}, and τ₂ and τ₁ are time constants that control growth and displacement, respectively. All constants are functions of experimental parameters, and the experimental curves are well fitted with this equation, as shown in FIG. 2. The calibration curve (FIG. 2B) generated from the fits illustrates the quantitative capabilities of this method in the linear regime, with roughly 2 orders of magnitude in dynamic range. By choosing a lower concentration of the competitor (mM), targets at 0.1 nM or lower concentrations were detected. It is also important to note that detection is performed in the transient regime such that equilibrium need not be reached in order to perform quantitative analysis, unlike other approaches.

In a more realistic scenario in which there are more species present than the target and competitor, CDDM has an additional advantage. Any species that has equal or lower affinity than the competitor (i.e. an equal or greater degree of mismatch destabilization from the probe) cannot cause displacement of the competitor; therefore, a properly chosen competitor acts as a “filter” for lower affinity species. Displacement indicates that there must be a higher affinity species present in the sample, i.e. the target. It has been shown that a three-component computational model is sufficient to describe kinetic capture in the general case.

Research Methodology

To simulate heteroplasmy, selected mutations and flanking sequences (as described below) are amplified by the polymerase chain reaction (PCR) and cloned into standard recombinant DNA vectors (e.g, TOPO). Both wild type and mutant sequences are cloned and the resulting recombinant plasmids are quantified and used in mixing experiments to determine sensitivity of heteroplasmy detection. Appropriate statistical methods are utilized to determine the accuracy of heteroplasmy determination, as well as standard deviation of quantitative data. If the results of testing show adequate accuracy (˜90%) within the required dynamic range (>20), they are followed by experiments on de-identified patient samples.

Characterization of CDDM on a Single Mutation Locus.

CDDM is characterized using a frequent pathogenic mutation G11778A in mtDNA. Total DNA is extracted from blood samples (Quiagen DNA mini prep kit) and PCR amplified using published primer pair (Jiang 2007) under asymmetric conditions (0.5 uM/0.1 uM) to produce ssDNA targets within the range of 100-300 nM depending on amplification conditions.

TABLE 1

Representation of the probes and anti-probe
competitor. The folding melt temperature and
hybridization melt temperatures are
given for reference.

| | | |
|---|---|---|
| sequence | Tm(C) | f(C) |

| | | | |
|---|---|---|---|
| Wt probe | 83.9 | 47.3 | 5′-gca aactcaaact |
| | | | acgaacgcac tcacagtcGc |
| | | | atcataatcc tctctcaagg |
| | | | acttcaaa (SEQ ID NO: 3) |
| SNP probe | 83.0 | 50.9 | 5′-gca aactcaaact |
| | | | acgaacgcac tcacagtcAc |
| | | | atcataatcc tctctcaagg |
| | | | acttcaaa (SEQ ID NO: 4) |
| Competitor | 83.9 | 51.1 | 5′-tttgaagt ccttgagaga |
| wt Anti- | | | ggattatgat gCgactgtga |
| Probe | | | gtgcgttcgt agtttgagtt |
| (11748 to | | | tgc (SEQ ID NO: 5) |
| 11808) | | | |
| wt | 93.0 | 47.3 | 5′-catcc aaaccccctg |
| Amplicon | | | aagcttcacc ggcgcagtca |
| (11655 to | | | ttctcataat cgcccacgga |
| 11866) | | | ctcacatcct cattactatt |
| | | | ctgcctagca aactcaaact |
| | | | acgaacgcac tcacagtCgc |
| | | | atcataatcc tctctcaagg |
| | | | acttcaaact ctactcccac |
| | | | taatagcttt ttgatgactt |
| | | | ctagcaagcc tcgctaacct |
| | | | cgccttaccc cccactatta |
| | | | acctactg (SEQ ID NO: 6) |

Resulting amplicons are 115 bp in length. The short amplicons are better suited for surface sensing than longer ones because they minimize secondary structure effects and have less steric hindrances when reacting with the surface bound probes. An Alexa488-labeled competitor based on the wt sequence with central position of the SNP (G11778A) is synthesized and mixed with PCR reaction without further purification. The resulting mixture is applied to the sensing surface by utilizing our real-time hybridization platform with hybridization volumes 25-100 μL. Preparation of the sensing surface: silanization, spotting, and blocking follow published procedures (Bishop 2007). Real-time monitoring of hybridization reaction is performed, and background signal subtraction and signal normalization will be performed (Bishop 2007). Fitting of the competitor kinetic curve from each spot is performed as described as above, based upon an extended, three-component model using wt and SNP concentrations as parameters (which determine the degree of heteroplasmy).

Due to some variability in custom spotting, calibration of each sensing zone is accomplished by pre-hybridizing 10 nM solution of the competitor to the array in the absence of the sample DNA, followed by melting the hybrids and washing just prior to the experiment in which the sample is mixed with the competitor. Experimental fluorescence intensities are normalized to these positive control values. Upon completion of the R21 with probes designed for all spots on the array, array spotting can be performed commercially (i.e. Agilent or Nimblegen), in which case spotting variability is low enough that positive controls can be used simultaneously with sample assay, eliminating the extra calibration step.

Two sensing zones are designed (and used in duplicate): one perfectly matched to the wt sequence and the other perfectly matched to the point mutation. 60mer probes are used with the mutation spot mapped approximately to the center of the probes (Table 1). Using longer probes and high temperature hybridization (up to about 76° C.) can reduce non-specific binding to the zones, thus increasing specificity of target capture, limiting specific binding in this case to just wt sequence (both target and competitor) and mutated sequence (SNP target). In order to predict possible results and times of the assay, computer simulations of hybridization reactions with different species composition using thermodynamic parameters of the probes and competitor were performed.

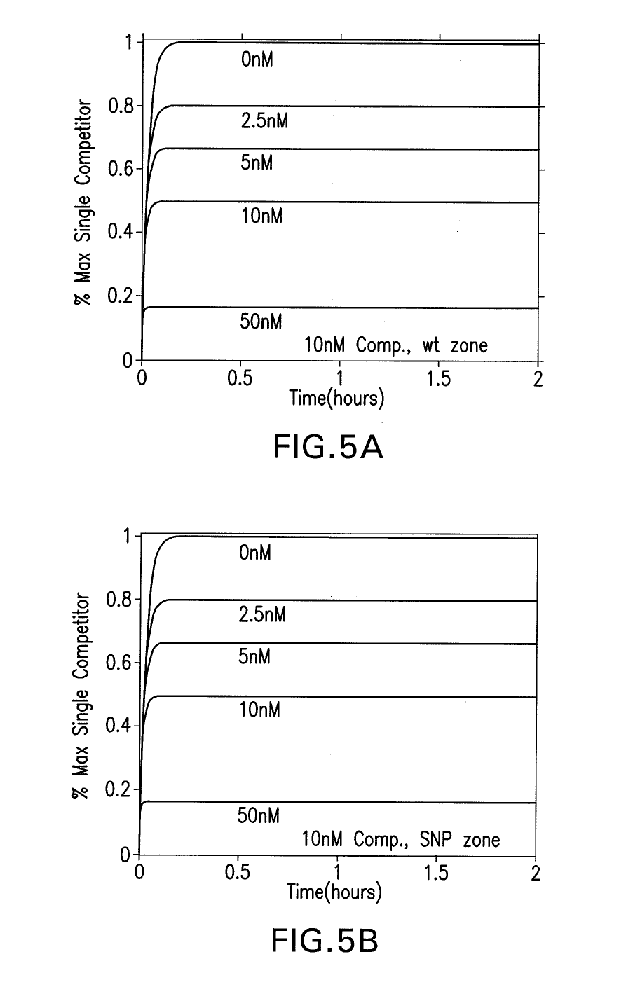

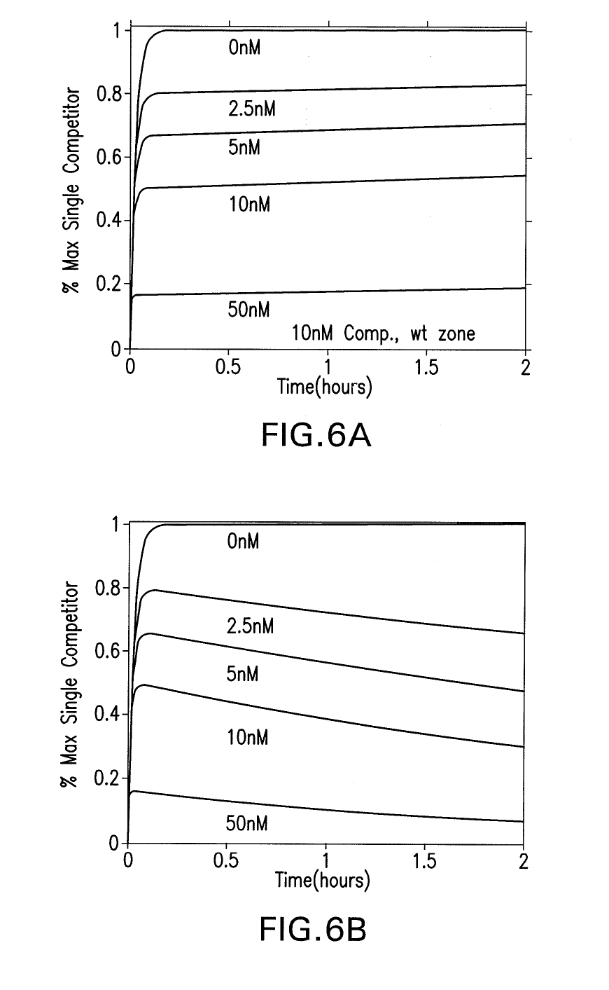

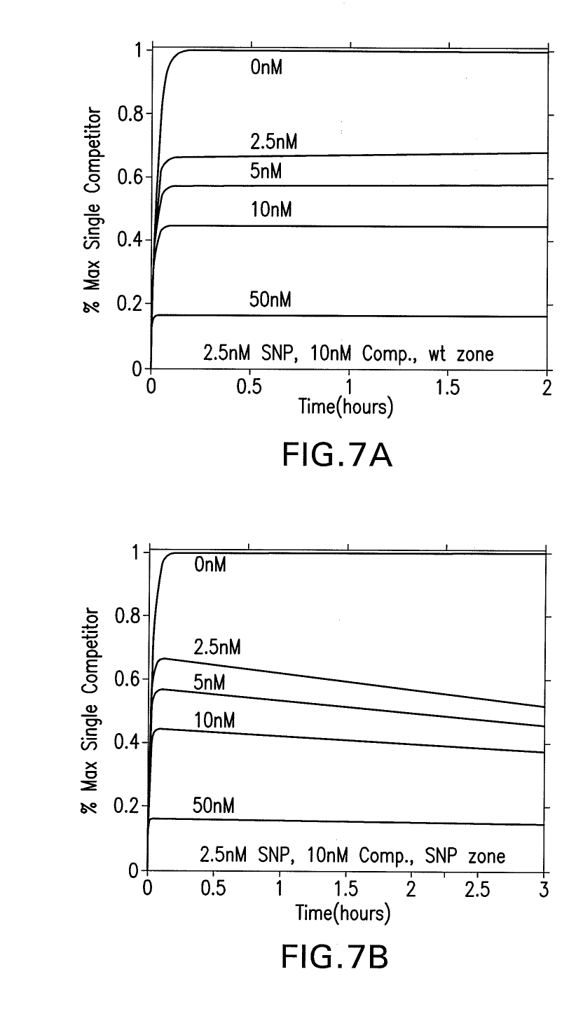

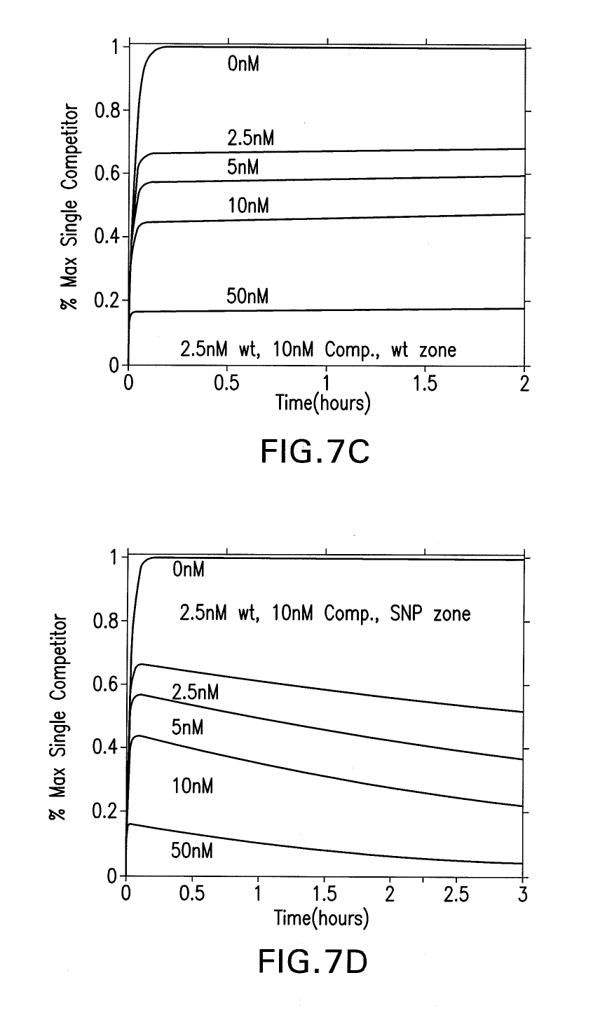

Three different scenarios of the assay can occur: the sample is homogeneous with wt sequence, the sample is homogeneous with the SNP sequence, or the sample is heterogeneous with varying heteroplasmy compositions. Simulated results of the homo-wt experiment are presented in FIG. 2. In both zones decrease in the maximal signal is observed, because there is partitioning (not competitive displacement) between labeled (spiked) and unlabeled (sample) ssDNA for binding with the probes. However, in both zones the binding curves rise monotonously with no displacement of the labeled species. There is an option of quantifying concentration of the wt species by the level of plateau of the signal, but just qualitative examination of the fluorescence curves indicates the desired answer: there is no mutant in the sample. The second scenario is realized when the sample is represented by homogeneous mutated species. In this case the binding curve on the wt zone reaches close to the level of the positive control (i.e. competitor applied alone without the sample) although initial binding is slowed down by transient competition (in which displacement of the mutant species occurs), while response on the SNP spot exhibits displacement of the competitor by the target ssDNA, and this response can be quantified with respect to the sample DNA concentration (FIG. 5). The general case is presented when both wt and SNP species are present in the sample. The wt spot signal behavior indicates the presence of the wt target, which can be quantified by measuring decrease of the plateau signal, while competitor displacement from the SNP spot indicates presence of the SNP species in the sample, which can be quantified using appropriate calibration of the signal maximum (FIG. 6). By using a single concentration of competitor at 10 nM, relative concentrations of sample species in the range of 2.5-50 nM can be quantified with desired dynamic range of 20 (i.e. 5% heteroplasmy).

Scaling Up CDDM to Interrogate Multiple Mutations.

The array can be expanded to interrogate 13 amplicons encompassing 15 reported pathogenic mutations in the human mitochondrial genome (Jiang 2007). Design of the primers follow published sequences (Jiang 2007). Design of the array spots and corresponding competitors follow guidelines outlined above. Physical separations between zones binding the same competitor species (wt and SNP) are optimized: if the distance between the spots is relatively large they react independently.

There are definite advantages in utilizing 60mers and high temperature hybridizations: majority of secondary structure motifs in probes, competitors, and targets are resolved and non-specific (partially complementary) hybrids both in solution and on the surface are destabilized. Also, the rate of surface capture is higher, so the time of the experiment is shorter. Although diffusion of the longer DNA competitors to the surface is slower, it may not reflect in the observed kinetics of binding considering the relatively high concentrations of targets and competitors.

Comparative Validation of CDDM Versus Single Nucleotide Extension Method.

Single nucleotide extension (SNE) assays are developed for the detection of major pathological mutations in the mitochondrial genome. The CDDM approach can achieve the same or better levels of accuracy and dynamic range but in a more rapid and cost-effective manner. Therefore, comparative studies are performed between CDDM and SNE using de-identified patient samples that have been sequence verified by direct sequencing.

A battery of PCR amplified patient samples are assayed on the CDDM and SNE platforms by the same technician, keeping track of assay time and reagent usage. The platforms are further characterized for the accuracy of calls as compared to the sequencing results and the total dynamic range obtained per locus. Reproducibility of the platforms are characterized by making multiple assays of a given sample.

### Example 3

### Kinetics of Multiplex Hybridization: Mechanisms and Implications

Using computer simulations and real-time experimental results, effects of multiplex reactions on a single sensing zone of an array have been demonstrated, which can be a leading factor in erroneous interpretation of experimental data. It is shown herein that a simplified three-component kinetic model can describe DNA sensing in a complex sample milieu. It is shown that, by analyzing the real-time hybridization kinetics of a non-target species, quantitative analysis of unlabeled targets of interest within a broad dynamic range of concentrations can be performed.

### Introduction

Use of DNA microarray-based analysis has quickly expanded in genetic research and in various genomic applications since its introduction in the early 1990s (Levicky 2005). Indeed, microarrays offer geneticists the opportunity to analyze massive amounts of data (up to the whole genome) in the course of a single experiment. The primary goal of microarray-based methodologies is to answer two basic questions: what, and how much; i.e., to determine the quantitative and qualitative (sequence-specific) composition of nucleic acids in the sample). It is herein shown that the kinetic approach, which requires a paradigm shift in microarray experimentation, can resolve many issues associated with molecular recognition in complex samples. In fact, (Fish 2007; Bishop 2007; Bishop 2008) the time-course of competitive displacement phenomena in multiplex environments, with excellent agreement to simulation results, has been shown experimentally. It is further understood that sequence effects are reflected in the corresponding rate constants (Tawa 2005) (in the approximation used here, the dissociation rate constants).

Here, how changes in sample composition modify the kinetics of hybridization are studied. Using a multi-component kinetic model of hybridization, in which the hybridization of all DNA sequences to a single sensing zone are intertwined, simulations varying the number and concentration of DNA species are performed. It is shown that, even in this complex environment, the kinetics of hybridization can be adequately described by considering only three components—the perfectly matched target, a high affinity non-target (competitor), and low affinity non-target (apparent background). This approximation significantly simplifies the development of analysis methods. The simulation work is then extended to an experimental setup where the hybridization kinetics of the competitor species are tracked in real-time and it is shown how it is affected by the presence of a perfectly matched target plus two background species with small sequence variation and varying concentrations. Using both simulation and experimental results, the discussion is extended from the effects of cross-hybridization of background species to the use of the real-time hybridization curve of the competitor to quantitatively assess the concentration of a perfectly matched target under nonequilibrium conditions in the context of the three-component model, even in the presence of multiple background species.

### Theory

A simplification to the more general model of the chemical reaction of N different species to a single probe species was considered (Horn 2006; Bishop 2006):

$$\frac{d {B}_{i} ( t )}{d t} = {k}_{a , i} {C}_{i} ( t ) [ {R}_{T} - {\sum }_{j = 1}^{N} {B}_{j} ( t ) ] - {k}_{d , i} {B}_{i} ( t ) , \qquad ( 1 )$$

where Bᵢ(t) represents bound surface concentrations, k_{a,i} the association rate constant for each reaction, Cᵢ(t) the solution concentration, RT the concentration of probe, and k_{d,i} the dissociation rate constants. This environment is one in which all species can compete for the same probe site, but at equilibrium the highest affinity species will partially displace all others, with dynamic range at equilibrium determined by the relative dissociation rates and concentrations of species (Bishop 2006). This model disregards surface-specific effects: electrostatic repulsion and probe accessibility (Erickson 2003; Peterson 2002). Relative target to probe orientation and its effects on nucleation rates is also disregarded (Hagan 2004) as well as cross-hybridization of targets in solution (Home 2006) and secondary structure effects (Gao 2006) (although the latter effects may be described by the use of effective concentrations (Bishop 2007)). This model can be readily extended to include these effects, but this simplification is sufficient to study the essential features of competition in multiplex hybridization.

In a typical experiment with end-point measurement, the quantity being measured at each spot is total fluorescence

$$F ( t ) = c {\sum }_{i = 1}^{N} {B}_{i} ( t ) , \qquad ( 2 )$$

where t is the hybridization time, c is a proportionality constant, and it is assumed that all species are identically fluorescently labeled. In a real-time experiment, F(t) is measured continuously at discrete time points.

Mass transport can reasonably be incorporated using the two-compartment model, where the concentration Ci(t) in the lower compartment is described by (Myszka 1998; Chechetkin 2007)

$$\frac{V}{S} \frac{d {C}_{i} ( t )}{d t} = - {k}_{a , i} {C}_{i} ( t ) [ {R}_{T} - {\sum }_{j = 1}^{N} {B}_{j} ( t ) ] + {k}_{d , i} {B}_{i} ( t ) + {k}_{M} [ {C}_{i} - {C}_{i} ( t ) ] , \qquad ( 3 )$$

where the Cᵢ values represent constant concentrations in the upper compartment, V is the volume of the lower compartment, S is the surface area intersecting the two compartments, and kM represents the diffusion of target across the interface.

As a simplified basis for analysis of microarray data, a simplified three-component model consisting of the complementary target, the highest affinity mismatch (a competitor), and a composite of all other interacting species with lower affinity (background) is also considered,

$$\begin{matrix}\frac{d {B}_{t}}{d t} = {k}_{a , t} {C}_{t} ( t ) [ {R}_{T} - {B}_{t} ( t ) - {B}_{c} ( t ) - {B}_{b} ( t ) ] - {k}_{d , t} {B}_{t} ( t ) , & ( 4 ) \\ \frac{d {B}_{c}}{d t} = {k}_{a , c} {C}_{c} ( t ) [ {R}_{T} - {B}_{t} ( t ) - {B}_{c} ( t ) - {B}_{b} ( t ) ] - {k}_{d , c} {B}_{c} ( t ) , & ( 5 ) \\ \frac{d {B}_{b}}{d t} = {k}_{a , b} {C}_{b} ( t ) [ {R}_{T} - {B}_{t} ( t ) - {B}_{c} ( t ) - {B}_{b} ( t ) ] - {k}_{d , b} {B}_{b} ( t ) , & ( 6 )\end{matrix}$$

where the appropriate modifications of the lower compartment concentrations also need to be made.

### Materials and Methods

### Simulation

The analytical description of multiplex hybridization described in the previous section is to perform virtual experiments with N≦7. It is assumed that the association rates of all the species are equal (Sekar 2005), with value 10⁶ M⁻¹ s⁻¹. The dissociation rate for the primary target is set ^{tok}d,t⁼4.55×10⁻⁶ s⁻¹, the competitor is k_{d,c}=5×10⁻⁴ s⁻¹, and the background dissociation rates range from 7.5×10−4 s⁻¹ to 3.67×10⁻² s⁻¹. This range was chosen to simulate increasingly unstable targets. It should be pointed out that the dissociation rate constants and association rate constants chosen for the simulations are the same ones used during the fitting of the experimental data. Additionally, the probe concentration RT is set to 10⁻¹¹ Mm for the simulations and allowed to vary during the fit of experimental data. However, RT stayed within the range of 1×10⁻¹¹ to 5×10⁻¹¹ for all fits. In addition, the coefficient representing diffusion between the upper and lower compartments, kM, is set to 10⁻⁶ cm/s.

Simulations were performed by implementing custom code in MATLAB (The MathWorks, Natick, Mass.). The function ode15s was used as a differential equation solver. In performing the fits with the N=3 model, the fminsearch optimization routine was used.

### Experimental

Mixtures of four different 20-mer sequences: a target of interest (5′ to 3′ CGAGGGCAGCAATAGTACAC (SEQ ID NO: 1), perfect complement to the probe), a competitor (Cy-3 CGAGGGCAGCATTAGTACAC, SEQ ID NO: 2), a tandem mismatch (CGAGGGCAGCATAAGTACAC, SEQ ID NO: 7), and a three-base deletion plus insertion (CGAGGGCAGCAGTACACTTT, SEQ ID NO: 8). Only the competitor sequence is fluorescently labeled, and is therefore the only sequence detected by our fluorescence reader. In a previous article (Bishop 2007), it was shown that it is sufficient to monitor the kinetics of the competitor to assess the presence and concentration of the target sequence, hence allowing label-free detection of the target.

Real-time experiments are performed using a custom-built fluorescence detection setup (Bishop 2007). A 532-nm laser was end-fire-coupled into a quartz microscope slide which served as an optical waveguide and therefore produced an evanescent field responsible for fluorescence excitation at the surface. The slide also served as the sensing surface where immobilized probe sites are located and where hybridization takes place. The microscope slide is fixed to a custom heating unit that uses a computer-controlled Peltier heater to adjust the temperature of the hybridization. All experiments are performed at 30° C. A hybridization chamber was made by applying a PDMS gasket to the quartz slide. After dispensing the hybridization solution into the inner boundary of the gasket, a glass slide is then placed on top of the gasket to complete the chamber. Using a CCD camera (ST-7XMEI; Santa Barbara Instrument Group, Santa Barbara, Calif.) mounted above the sensing surface, a time-dependent fluorescence signal, proportional to Bc(t), is detected. Each frame captured by the camera is exposed for 2.5 s and then saved for postprocessing. A digital output signal from the camera shutter is used to modulate the laser output to reduce photobleaching during the time interval between acquisitions. Further experimental details, such as surface modification chemistry, target and probe preparation, probe immobilization, and normalization, can be found in the literature (Bishop 2006; Bishop 2007).

### Results

### Simulations

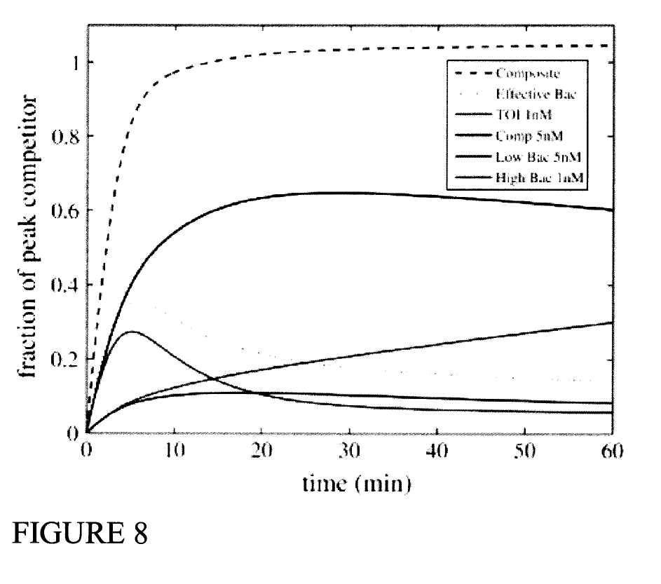

N=4 components are used to illustrate the kinetics of multicomponent hybridization: target, competitor, and two background species. Dissociation rates for the two background species are kd=7.5×10−4 s⁻¹ and kd=4.63×10⁻³ s⁻¹. FIG. 8 shows the individual hybridization curves, along with the composite hybridization curve that is observed assuming that all species are detected equally; it is clear that the composite curve gives very little indication as to the hybridization kinetics of the target species. To be consistent with other graphs, all hybridization curves are normalized against the maximum hybridization signal produced by the competitor species alone.

The hybridization curve of the target always increases monotonically; since the target is at a lower concentration than other species, it does not control the initial phase of hybridization (in this case, the competitor and low-affinity background control). In the presence of the target, the competitor will always be displaced (as shown here), while in the absence of the target, the competitor will be the highest affinity species and monotonically increase. As shown in the plot, the background species are displaced, but with very different kinetics. The low affinity background is at higher concentration, so it initially grows rapidly, but, because of its low affinity, it is quickly displaced. However, the high affinity background (at lower concentration) grows more slowly and is displaced more slowly. Even in a complex environment, the equilibrium distribution can be predicted via thermodynamic models, but it is not practical to measure experimentally. In the kinetic regime, the distribution of bound species is time-dependent, as should be clear here. Also shown in the figure is the composite background signal as determined by fitting with the three-component model.

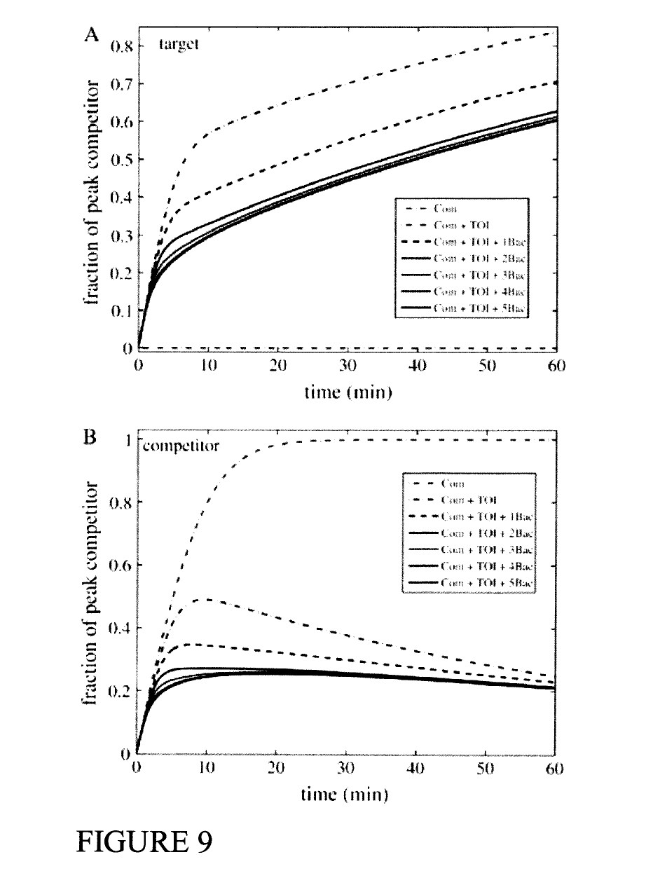

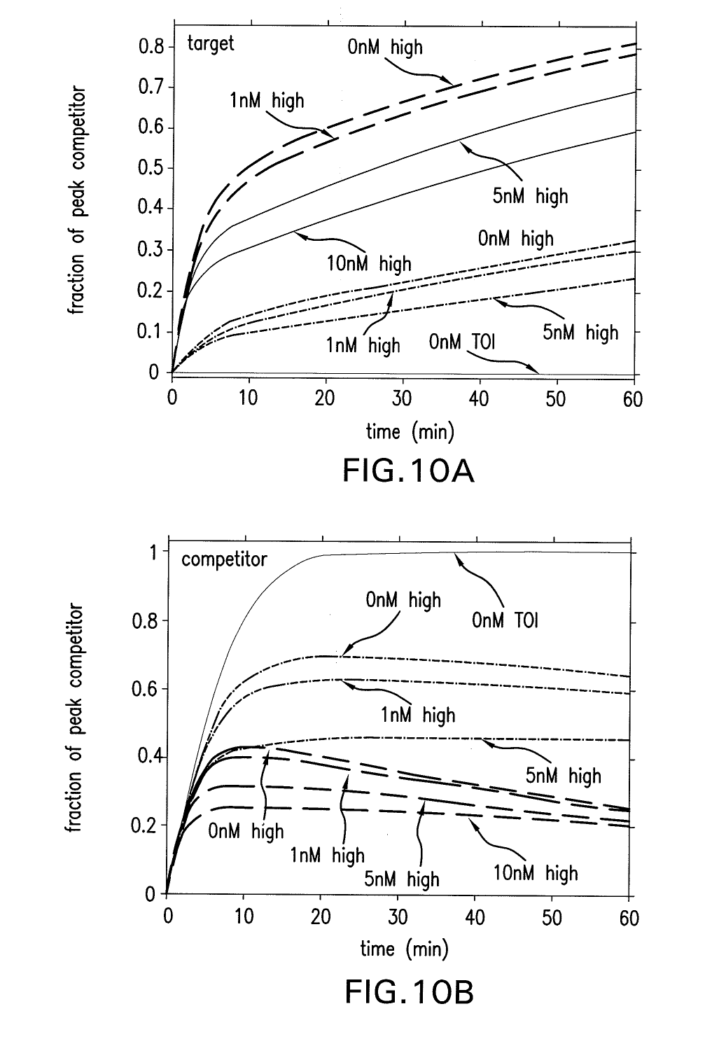

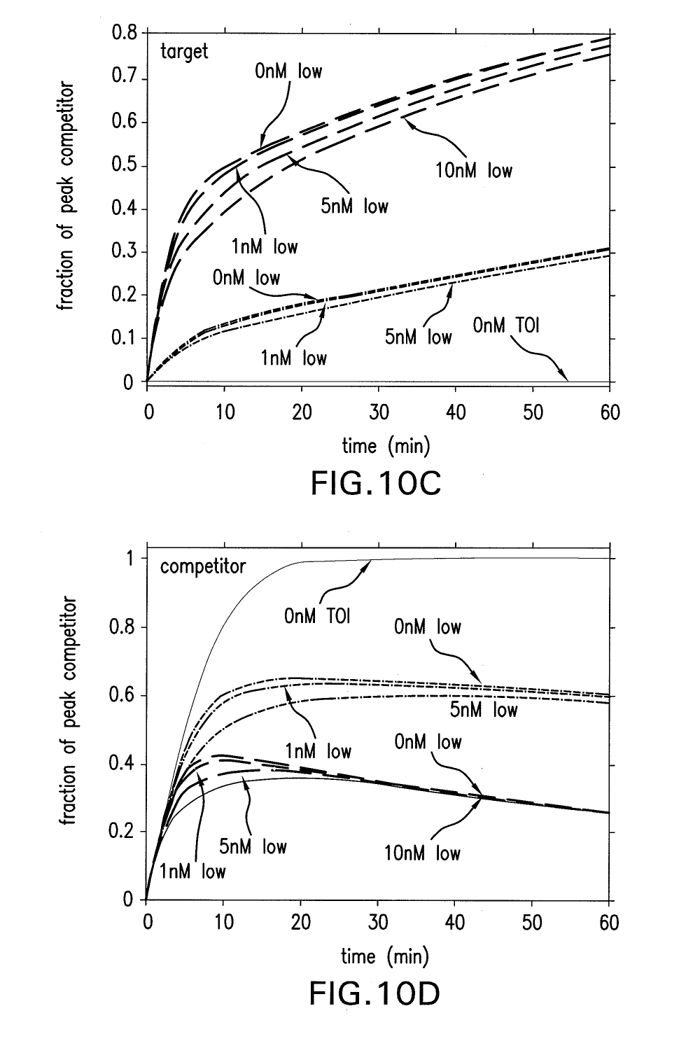

Next, how background hybridization affects the kinetics of the target and competitor was investigated. FIG. 9 shows the target and competitor hybridization curves as the target is added to the sample and as the number of background species increases from 0 to 5; the dissociation rates of the background increase from 7.5×10⁻⁴ s⁻¹ for the first background species to 3.67×10−2 s⁻¹ for the fifth. These results indicate dramatic effects on the hybridization kinetics of the target and competitor as reflected in the decrease in their bound concentrations. However, the influence on kinetics diminishes as the affinity of the background species decreases, i.e., there is very little change as the fifth background is added, showing that cross-hybridization diminishes significantly for species with high dissociation rates (for a fixed concentration).

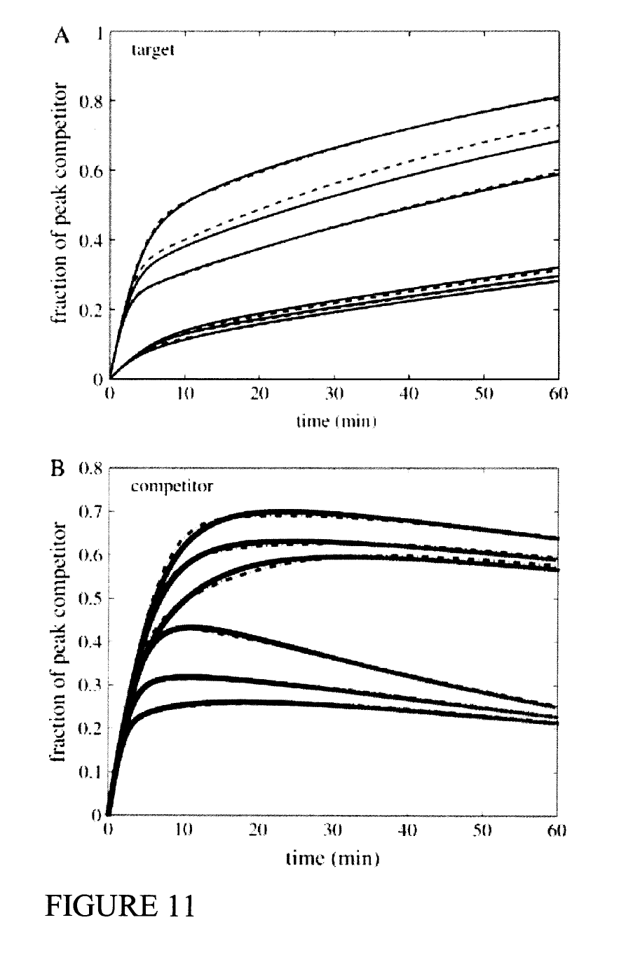

Using these five background species, simulations were performed to study how a change in the concentration of the high-affinity background species (kd=7.5×10⁻⁴ s⁻¹; see FIGS. 11, A and B) and the low-affinity background species (kd=4.63×10⁻³ s⁻¹; see FIGS. 11, C and D) affect the target and competitor. Two different target concentrations are shown (1 nM and 5 nM). These results show that as the concentration of the target increases, the hybridization rate of the competitor decreases rapidly. As the concentration of the high affinity background species increases, both the target and competitor are modified significantly. The lower-affinity background has less effect; the decrease in the hybridization rates of the target and competitor is less pronounced. However, these results show that even low-affinity species, at high enough concentrations, can still play a role in the signal that is acquired.

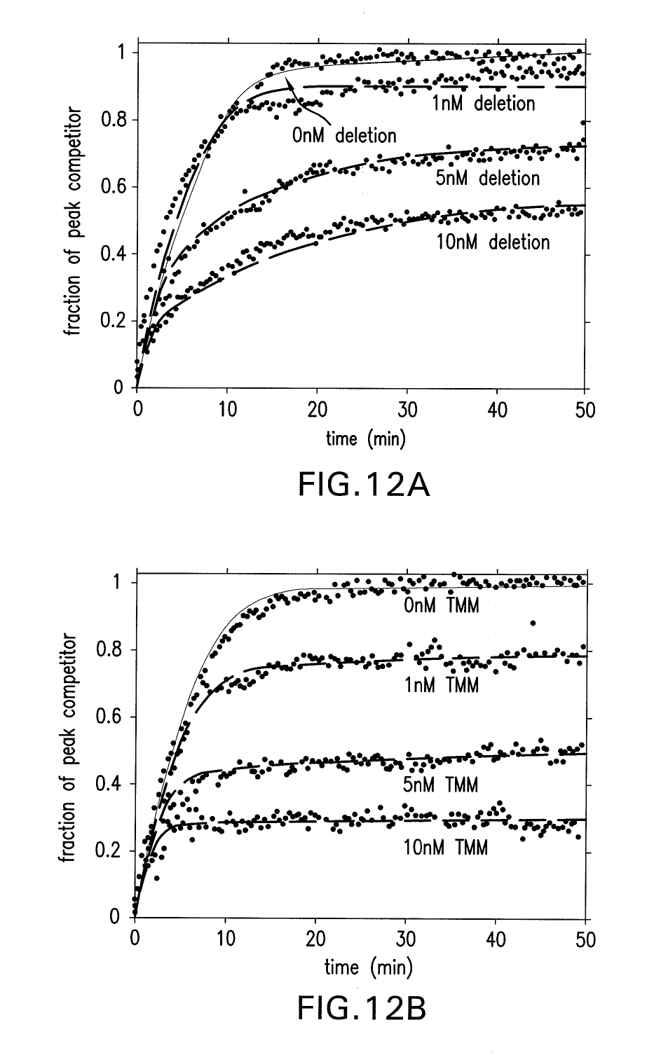

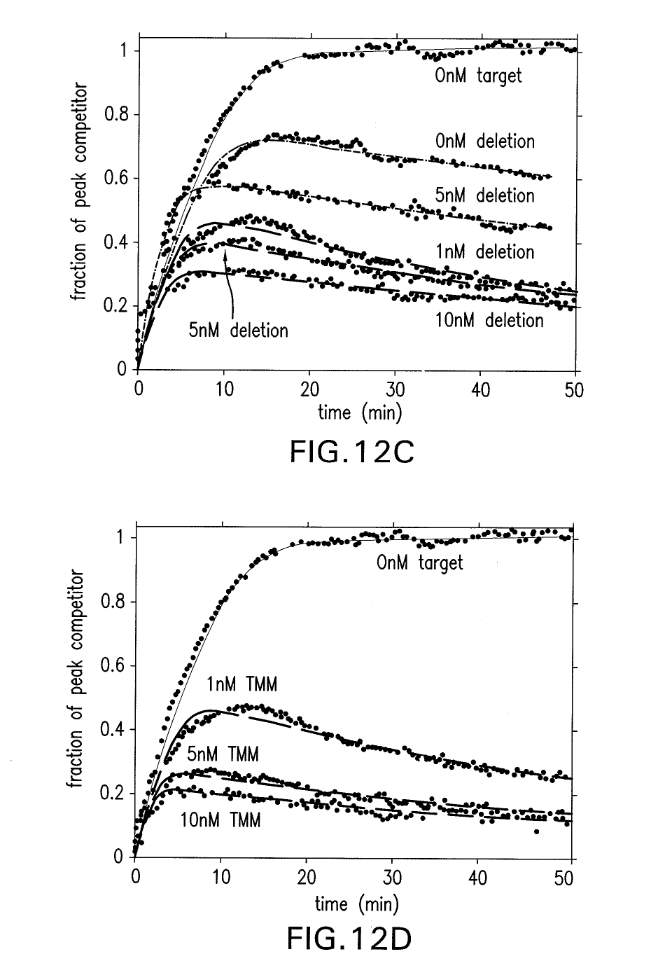

The above results motivate a major question for micro-array analysis: how many background species (i.e., model components) need to be accounted for during analysis? FIG. 12 shows that accounting for all possible combinations may not be necessary, where the hybridization of the target and competitor are plotted in the presence of two target concentrations and mixtures of five background species (details in Table 2). Using Eqs. 4-6, these hybridization curves have been fit, where Eq. 6 is assumed to be a composite of all remaining species, or in other words, the concentration and rate constants for a third component become apparent. For the fits in FIG. 4, it was assumed that the rate constants of both the target and competitor are known (via estimated thermodynamic parameters), and that the concentration of the competitor is known. The routine fits the competitor binding curve by adjusting the concentration of the target and the rate constants and concentration of the apparent background. Table 2 shows these fitted values; the fitted target concentration lies within 10% of the actual value used in the simulations.

Several interesting features are demonstrated by applying three-component fits to more complex cases (seven components in this example). First, even though the hybridization curve of the target is not considered in the fitting routine, the reconstructed target kinetics closely matches the simulated kinetics (as shown in FIG. 11A). Second, the kinetics of the apparent background (as represented by the effective dissociation coefficient) does not change significantly with varying background complexity. The effective k_{d,b} values vary by less than a factor of two, and fall within the middle of the dynamic range of the simulated values for the background species; however, the effective concentration of the background consistently tracks the total background concentration.

TABLE 2
Fitted Target of Interest (TOI) concentration and
background data from the simulation results

| Summed | TOI actual | TOI fit | Back. fit | Back. k_{d} |
|---|---|---|---|---|
| background (nM) | (nM) | (nM) | (nM) | (10⁻⁴ s⁻¹) |
| 4.0 | 1.0 | 1.0 | 1.8 | 15 |
| 5.0 | 1.0 | 1.1 | 3.0 | 11 |
| 9.0 | 1.0 | 1.1 | 5.3 | 14 |
| 4.0 | 5.0 | 5.0 | 3.1 | 11 |
| 9.0 | 5.0 | 5.3 | 7.1 | 11 |
| 14.0 | 5.0 | 5.5 | 12 | 8.8 |

Values given represent curves shown in FIG. 4 from highest to lowest fraction of peak competitor. The competitor concentration is 5 nM.

### Experimental

Experiments were first performed to verify the effect of background on the competitor in the absence of the target. FIG. 12A shows the hybridization curves of the competitor as the concentration of the deletion is increased, while FIG. 12B shows the hybridization of the competitor as the tandem mismatch (TMM) is increased. In both cases, the competitor is not displaced because it has a higher affinity than the two background species. Based on these results, it has also been verified that the deletion has a lower affinity than the TMM, but the deletion is still a significant source of background.

Fits of the experimental traces were performed using the three-component approximation described above, with the background concentration and rate constants free parameters, but with known competitor concentration and rate constants, and known target rate constants. In the three-component fit here, an apparent target concentration is obtained even though no target is present in solution. This concentration represents the lower limit of discrimination and is ˜10 fM for these experiments. This lower limit can be decreased by either measuring over a longer period of time or by improving the signal/noise ratio of the system.

In a microarray experiment, a researcher is searching for a target in the presence of many other species. FIG. 12C uses two different target concentrations and shows how, as the deletion concentration is increased in the mixture, the competitor hybridization is changed. Additionally, FIG. 12D shows how the competitor hybridization changes as the concentration of the TMM increases. The experimental data were fit using the same procedure described above, verifying that the three-component model works experimentally when a target of interest is present. Table 3 shows that the fits predict target concentrations within 25% of the experimental values. Similar to the result shown in FIGS. 12, A and B, the TMM decreases the hybridization rate of the competitor more than does the deletion, because of its higher relative affinity. It should be noted that at equimolar target and competitor concentration, the maximum value of the competitor (normalized to the maximum value of the competitor alone) is 0.5, so that the effects of background can be evaluated by deviation of the competitor peak below this value.

TABLE 3
Fitted Target of Interest (TOI) concentration and background
data from the experimental curves shown in FIG. 12

| Deletion | TMM | TOI actual | TOI fit | Back. fit | Back. k_{d} |
|---|---|---|---|---|---|
| (nM) | (nM) | (nM) | (nM) | (nM) | (10⁻⁴ s⁻¹) |
| 0 | 1 | 1.6 | 1.2 | 0.44 | 16 |
| 5 | 1 | 1.6 | 1.9 | 1.6 | 10 |
| 1 | 1 | 5 | 5.2 | 0.65 | 14 |
| 5 | 1 | 5 | 4.9 | 2.1 | 9.5 |
| 10 | 1 | 5 | 4.8 | 7.9 | 8.3 |
| 1 | 5 | 5 | 6.2 | 3.7 | 10 |
| 1 | 10 | 5 | 6.3 | 12 | 5.9 |

The competitor concentration is 5 nM.

### Discussion and Conclusion

Recently, the kinetics of hybridization of a primary target in the presence of a secondary mismatched species has been investigated using computational (Bishop 2007; Bishop 2006; Home 2006; Zhang 2005; Chetchetin 2007) and experimental (Fish 2007; Bishop 2007) approaches. These studies have made the following apparent:

1. The specificity of recognition is controlled by the competitive displacement of the lower-affinity species by the higher-affinity one;

2. The signature of this mechanism is anonmonotonic growth curve of the lower-affinity species;

3. Depending on the relative concentration and rate constants of the primary and secondary species, the latter can become a major contributor to the observed signal, especially in the transient regime;

4. The presence of the secondary species extends the time to reach equilibrium;

5. The maximum specificity is obtained at equilibrium; and

6. In the absence of the primary target, the secondary species can produce a signal comparable to the primary target alone.

These observations were generalized here to the more complex environment in which the interaction among N species (i.e., a target and N−1 background) must be considered, all of varying affinities to a given probe. The fundamental questions are then how the background affects the kinetics of the target hybridization and how the target can be properly quantified given the influence of the background. It was shown that the complex N component system can be reduced to a system of three components (the target plus two effective background components, one of which we term the competitor). The validity of this reductionist approach was validated by fitting simulated and experimental binding curves of samples with different compositions. This result opens the way to develop new analytical techniques for quantitative analysis. For example, it was shown that by analyzing the binding curve of a known secondary target, which is called a competitor, one can evaluate the concentration of a primary target in an unknown sample composition.

These results demonstrate several important advantages of the kinetic analytical approach discussed above. One is that false positive calls are practically eliminated. Indeed, in the absence of primary targets, the competitor binding curves grow monotonically, while in the presence of the primary targets, they display nonmonotonic behavior. Secondly, the detection approach employed eliminates fluorescent labeling of the sample, because target quantitation is based solely on the binding of the competitor (which itself must be fluorescently labeled). Third, by applying a three-component model to analyze experimental binding curves, expanded dynamic range of quantitations was observed: in this example it was determined to be 106. Lastly, the time of the experiment is greatly reduced: analysis is performed on transient binding curves, which eliminates requirements of reaching equilibrium.

In conclusion the kinetics of multiplex hybridization have been studied, and it has been shown that a simplified three-component kinetic model is sufficient to capture the dynamics. In fact, the three-component model provides remarkably good agreement with experimental results.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Throughout this application, various publications are referenced. The disclosures of these publications in their entireties are hereby incorporated by reference into this application in order to more fully describe the state of the art to which this pertains.
Although the invention has been described with reference to the presently preferred embodiments, it should be understood that various modifications can be made without departing from the spirit of the invention. Accordingly, the invention is limited only by the following claims.

Throughout this application, various publications are referenced. The disclosures of these publications in their entireties are hereby incorporated by reference into this application in order to more fully describe the state of the art to which this pertains.

### REFERENCES

Adey, N. B., M. Lei, M. T. Howard, J. D. Jensen, D. A. Mayo, D. L. Butel, S. C. Coffin, T. C. Moyer, D. E. Slade, M. K. Spute, A. M. Hancock, G. T. Eisenhoffer, B. K. Dalley, and M. R. McNeely. “Gains in sensitivity with a device that mixes microarray hybridization solution in a 25-mm-thick chamber.” Anal. Chem. 74:6413-6417 (2002).
Altug, H. and Vuckovic, J. “Polarization control and sensing with two-dimensional coupled photonic crystal microcavity arrays” Optics Letters 30:982-984 (2005).
Attridge, J. W., Daniels, P. B., Deacon, J. K., Robinson, G. A. and Davidson, G. P. “Sensitivity enhancement of optical immunosensors by the use of a surface plasmon resonance fluoroimmunoassay” Biosensors and Bioelectronics 6:201-214 (1991).
Berrar, D. P., W. Dubitzky, and M. Granzow, editors. “A Practical Approach to Microarray Data Analysis.” Kluwer Academic Publishing, New York (2003).
Bhanot, G, Louzoun, Y., Zhu, J. and DeLisi, C. “The importance of thermodynamic equilibrium for high throughput gene expression arrays” Biophys. J. 84:124-135 (2003).
Bianchi, N., Rustigliano, C., Tomassetti, M., Feriotto, G., Zorzato, F. and Gambari, R. “Biosensor technology and surface plasmon resonance for real-time detection of HIV-1 genomic sequences amplified by polymerase chain reaction,” Clinical and Diagnostic Virology 8:199-208 (1997).
Bishop, J., Blair, S. and Chagovetz, A. M. “A competitive kinetic model of nucleic acid surface hybridization in the presence of point mutants” Biophys. J. 90:831-840 (2006).
Bishop, J., Chagovetz, A. M. and Blair, S. “Competitive displacement of DNA during surface hybridization” Biophys. J. 92:L10-12 (2007).
Bishop, J., S. Blair, and A. Chagovetz. “Convective flow effects on DNA biosensors.” Biosens. Bioelectron. 22:2192-2198 (2007).
Bishop, J., A. Chagovetz, and S. Blair “Competitive displacement: a sensitive and selective method for the detection of unlabeled molecules.” Opt. Express 15:4390-4397 (2007).
Bishop, J. S., A. M. Chagovetz and S. Blair “A sensitive method for label free molecular detection” Submitted to Optics Express (2007).
Blair, S. and Chen, Y. “Resonant-enhanced evanescent-wave fluorescence biosensing using cylindrical optical cavities” Applied Optics 40:570-582 (2001).
Boyd, R. W. and Heebner, J. E. “Sensitive disk resonator photonic biosensor,” Applied Optics-OT 40:5742-5747 (2001).
Chan, V., D. J. Graves, P. Fortina, and S. E. McKenzie. “Absorption and surface diffusion of DNA oligonucleotides at liquid! solid interfaces.” Langmuir 13:320-329 (1997).
Chechetkin, V. R. “Two-compartment model for competitive hybridization on molecular biochips.” Phys. Lett. A. 360:491-494 (2007).
Dai, H., M. Meyer, S. Stepaniants, M. Ziman, and R. Stoughton “Use of hybridization kinetics for differentiating specific from non-specific binding to oligonucleotide microarrays” Nucleic Acids Res. 30:e86-1-8 (2002).
Dandy, D. S., P. Wu, and D. W. Grainger. “Array feature size influences nucleic acid surface capture in DNA microarrays.” Proc. Natl. Acad. Sci. USA. 104:8223-8228 (2007).
Ditlbacher, H., Felidj, N., Krenn, J. R., Lambprecht, B., Leitner, A. and Aussenegg, F. R. “Electromagnetic intereaction of fluorophores with designed 2D silver nanoparticle arrays” Applied Physics B 73:373 (2001).
Elanko, N. and Jeffery, S. “Mutation analysis of PTPN11 in Noonan syndrome by WAVE. Methods Mol. Med. 126:97-111 (2006).
Erickson, D., X. Liu, U. Krull, and D. Li. “Electrokinetically controlled DNA hybridization microfluidic chip enabling rapid target analysis”. Anal. Chem. 76:7269-7277 (2004).
Erickson, D., D. Li, and U. Krull. “Modeling of DNA hybridization kinetics for spatially resolved biochips.” Anal. Biochem. 317: 186-200 (2003).
Fish, D. J., M. T. Home, R. P. Searles, G. P. Brewood, and A. S. Benight. “Multiplex SNP discrimination.” Biophys. J. 92:L89-L91 (2007).
Gao, Y., L. K. Wolf, and R. M. Georgiadis. “Secondary structure effects on DNA hybridization kinetics: a solution versus surface comparison.” Nucleic Acids Res. 34:3370-3377 (2006).
Hagan, M. F., and A. K. Chakraborty. “Hybridization dynamics of surface immobilized DNA.” J. Chem. Phys. 120:4958-4968 (2004).
Lee, N. H., and A. I. Saeed. “Microarrays: an overview.” Methods Mol. Biol. 353:265-300 (2007).
Hall T A, Budowle B, Jiang Y, Blyn L, Eshoo M, Sannes-Lowery K A, Sampath R, Drader J J, Hannis J C, Harrell P, Samant V, White N, Ecker D J, Hofstadler S A “Base composition analysis of human mitochondrial DNA using electrosspray ionization mass spectrometry: a novel tool for the identification and differentiation of humans.” Anal Biochem. 344(1):53-69 (Sep. 1, 2005).
Halperin, A., Buhot, A. and Zhulina E. B. “Sensitivity, specificity, and the hybridization isotherms of DNA chips.” Biophys. J. 86:718-730 (2004).
Halperin, A., Buhot, A. and Zhulina, E. B. “On the hybridization isotherms of DNA microarrays: the Langmuir model and its extensions.” J. Phys.: Condens. Matter. 18:S463-S490 (2006).
Held, G. A., G. Grinstein, and Y. Tu. “Relationship between gene expression and observed intensities in DNA microarrays—a modeling study.” Nucleic Acids Res. 34:e70 (2006).
Henry, M. R., Stevens, P. W., Sun, J. and Kelso, D. M. “Real-time measurements of DNA hybridization on microparticles with fluorescence resonance energy transfer,” Analytical Biochemistry 276:204-214 (1999).
Home M T, Fish D J, Benight A S “Statistical thermodynamics and kinetics of DNA multiplex hybridization reactions” Biophys J. 91(11):4133-53 (2006).
Ilic, B., Yang, Y., Aubin, K., Reichenbach, R., Krylov, S. and Craighead, H. G. “Enumeration of DNA molecules bound to a nanomechanical oscillator” Nano Letters 5:925-929 (2005).
Jiang Y, Hall T A, Hofstadler S A, Naviaux R K “Mitochondrial DNA mutation detection by electrospray mass spectrometry” Clin Chem. February; 53(2):195-203 Epub (2006 Dec. 7)
Kim, J., S. Doose, H. Neuweiler, and M. Sauer “The initial step of DNA hairpin folding: a kinetic analysis using fluorescence correlation spectroscopy” Nucleic Acid Res. 34:2516-2527 (2006).
Kleibl, Z., O. Havranek, and J. Prokopcova “Rapid detection of CAA/CAG repeat polymorphism in the AIB1 gene using DHPLC.” J Biochem Biophys Methods. Epub ahead of print—(2006).
Lee, H. J., A. W. Wark, Y. Li, and R. M. Corn “Fabricating RNA microarrays with RNA-DNA surface ligation chemistry.” Anal Chem. 77(23):7832-7 (2005).
Levicky, R., and A. Horgan. “Physicochemical perspectives on DNA microarray and biosensor technologies.” Trends Biotechnol. 23: 143-149 (2005).
Li, Y., A. W. Wark, H. J. Lee, and R. M. Corn. “Single-nucleotide polymorphism genotyping by nanoparticle-enhanced surface plasmon resonance imaging measurements of surface ligation reactions.” Anal Chem. 78(9):3158-64 (2006).
Liedberg, B., Nylander, C. and Lundström, I. “Surface plasmon resonance for gas detection and biosensing,” Sensors and Actuators 4:299-304 (1983).
Liu, Y. and Blair, S. “Fluorescence enhancement from an array of sub-wavelength metal apertures” Optics Letters 28:507-509 (2003).
Liu, Y., Bishop, J., Williams, L., Blair, S. and Herron, J. N. “Biosensing based upon molecular confinement in metallic nanocavity arrays” Nanotechnology 15:1368-1374 (2004).
Livshits, M. A., and A. D. Mirzabekov. “Theoretical analysis of the kinetics of DNA hybridization with gel-immobilized oligonucleotides.” Biophys. J. 71:2795-2801 (1996).
Malicka, J., Gryczynski, I. and Lakowicz, J. R. “DNA hybridization assays using metal-enhanced fluorescence” Biochemical and Biophysical Research Communications 306:213-218 (2003).
Markham, N. R. and M. Zuker “DINAMelt web server for nucleic acid melting prediction.” Nucleic Acids Res. 33:W577-W581 (2005).
Marotta, R., J. Chin, A. Quigley, S. Katsabanis, R. Kapsa, E. Byme, and S. Collins “Diagnostic screening of mitochondrial DNA mutations in Australian adults 1990-2001.” Intern Med J. 34(1-2):10-9 (2004).
Moerner, W. E. and Fromm, D. P. “Methods of single-molecule fluorescence spectroscopy and microscopy.” Review of Scientific Instruments 74:3597-3619 (2003).
Myszka, D. G., X. He, M. Dembo, T. A. Morton, and B. Goldstein. “Extending the range of rate constants available from BIACORE: interpreting mass transport-influenced binding data.” Biophys. J. 75:583-594 (1998).
Ngeh-Ngwainbi, J., Suleiman, A. A. and Guilbault, G. G. “Piezoelectric crystal biosensor S” Biosensors & Bioelectronics 5:13-26 (1990).
Oberacher, H., H. Niederstatter, and W. Parson “Liquid chromatography-electrospray ionization mass spectrometry for simultaneous detection of mtDNA length and nucleotide polymorphisms.” Int J Legal Med. 121(1):57-67 (2007).
Oh, S. J., B. J. Hong, K. Y. Choi, and J. W. Park. “Surface modification for DNA and protein microarrays. OMICS:” J. Integr. Biol. 10:327-343 (2006).
Okamoto, T., Yamaguchi, I. and Kobayashi, T. “Local plasmon sensor with gold colloid monolayers deposited upon glass substrates,” Optics Letters 25:372-374 (2000).
Okoniewski, M. J., and C. J. Miller. “Hybridization interactions between probesets in short oligo microarrays lead to spurious correlations.” BMC Bioinformatics. 7:276 (2006).
Peterson, A. W., L. K. Wolf, and R. M. Georgiadis. 2002. Hybridization of mismatched or partially matched DNA at surfaces. J. Am. Chem. Soc. 124:14601-14607.
Plowman, T. E., Reichert, W. M., Peters, C. R., Wang, H. K., Christensen, D. A. and Herron, J. N. “Femtomolar sensitivity using a channel-etched thin film waveguide fluoroimmunosensor” Biosensors and Bioelectronics 11: 149-160 (1996).
Plum, G. E. and K. J. Breslauer “Fluorescence energy transfer monitored competitive equilibria of nucleic acids: applications in thermodynamics and screening.” Biopolymers 61:214-223 (2002).
Podder, M., W. J. Welch, R. H. Zamar, and S. J. Tebbutt. “Dynamic variable selection in SNP genotype autocalling from APEX microarray data.” BMC Bioinformatics 30:7:521 (2006).
Provenzano, M., and S. Mocellin. “Complementary techniques: validation of gene expression data by quantitative real time PCR.” Adv. Exp. Med. Biol. 593:66-73 (2007).
Reichert, W. M. “Evanescent detection of adsorbed protein films: assessment of optical considerations for absorbance and fluorescence spectroscopy at the crystal solution and polymer solutions interfaces,” Critical Reviews in Biocompatability 5:173 (1989).
Riccelli P V, Merante F, Leung K T, Bortolin S, Zastawny R L, Janeczko R, Benight A S. “Hybridization of single-stranded DNA targets to immobilized complementary DNA probes: comparison of hairpin versus linear capture probes.” Nucleic Acids Res. 15; 29(4):996-1004 (2001).
Riccelli, P V, Hall, T S, Pancoska P, Mandell K E, Benight A S. “DNA sequence context and multiplex hybridization reactions: melting studies of heteromorphic duplex DNA complexes.” J Am Chem. Soc. 125(1):141-50 (Jan. 8, 2003).
Rindzevicius, T., Alayerdyan, Y. Dahlin, A., Hook, F., Sutherland, D. S. and Kall, M. “Plasmonic sensing characteristics of single nanometric holes,” Nano Letters 5:2335-2339 (2005).
Roederer, E. and Bastiaans, G. J. “Microgravimetric immunoassay with piezoelectric crystals,” Analytical Chemistry 55: 2333-2336 (1983).
Rudi, K., M. Zimonja, and B. Skanseng. “Quencher extension for single nucleotide polymorphism quantification in bacterial typing and microbial community analyses.” Methods Mol. Biol. 345:111-7 (2006).
Sekar, M. M. A., W. Bloch, and P. M. S. John. “Comparative study of sequence-dependent hybridization kinetics in solution and on microspheres.” Nucleic Acids Res. 33:366-375 (2005).
Sorokin, N. V., D. Y. Yurasov, A. L. Cherepanov, J. M. Kozhekbaeva, V. R. Chechetkin, O. A. Gran, M. A. Livshits, T. V. Nasedkina, and A. S. Zasedatelev. “Effects of external transport on discrimination between perfect and mismatch duplexes on gel-based oligonucleotide microchips.” J. Biomol. Struct. Dyn. 24:571-578 (2007).
Situma, C., M. Hashimoto, and S. A. Soper. “Merging microfluidics with microarray-based bioassays.” Biomol. Eng. 23:213-231 (2006).
Stimpson, D. I., Hoijer, J. V., Hsieh, W. T., Jou, C., Gordon, J., Theriault, T., Gamble, R., and Baldeschwieler, J. D. “Real-time detection of DNA hybridization and melting on oligonucleotide arrays by using optical waveguides” Proceedings of the National Academy of Science 92:6379-6383 (1995).
Storhoff, J. J., Marla, S. S., Garimella, V. and Mirkin, C. A. “Labels and detection methods” Springer 147-174 (2005).
Taitt, C. R., G. P. Anderson, and F. S. Ligler. “Evanescent wave fluorescence biosensors.” Biosens. Bioelectron. 20:2470-2487 (2005).
Taton, T. A., Mirkin, C. A. and Letsinger, R. L. “Scanometric DNA array detection with nanoparticle probes” Science 289:1757-1760 (2000).
Tawa, K., D. Yao, and W. Knoll. “Matching basepair number dependence of the kinetics of DNA-DNA hybridization studied by surface plasmon fluorescence pectroscopy.” Biosens. Bioelectron. 21:322-329 (2005).
Tsujikawa, K., M. Tsujikawa, H. Watanabe, N. Maeda, Y. Inoue, T. Fujikado, and Y. Tano. “Allelic homogeneity in Avellino corneal dystrophy due to a founder effect.” J Hum Genet. 52(1):92-7 (2007).
Vaisocherova, H., Zitova, A., Lachmanova, M., Stepanek, J., Karlikova, S., Liboska, R., Rejman, D., Rosenberg, I. and Homola, J. “Investigating oligonucleotide hybridization at subnanomolar level by surface plasmon resonance biosensor method.” Biopolymers 82: 394-398 (2005).
Wang, Y., C. Barbacioru, F. Hyland, W. Xiao, K. L. Hunkapiller, J. Blake, F. Chang, C. Gonzalez, L. Zhang, and R. R. Samaha. “Large-scale real-time PCR validation on gene expression measurements from two commercial long-oligonucleotide microarrays.” BMC Genomics. 7:59 (2006).
Wark, A. W., Lee, H. J. and Corn, R. M. “Long-range surface plasmon resonance imaging for bioaffinity sensors,” Analytical Chemistry 77:3904-3907 (2005).
Weis, B. K. “Standardizing global gene expression analysis between laboratories and across platforms.” Nat. Methods. 2:351-356 (2005).
Wong, L. J. “Comprehensive molecular diagnosis of mitochondrial disorders: qualitative and quantitative approach.” Ann N Y Acad Sci. 1011:246-58 (2004).
Wong, L. J. and R. G. Boles. “Mitochondrial DNA analysis in clinical laboratory diagnostics.” Clin Chim Acta. 354(1-2):1-20 (2005).
Wren, J. D., A. Kulkarni, J. Joslin, R. A. Butow, and R. G. Harold. 2002. Cross-hybridization on PCR-spotted microarrays. IEEE Eng. Med. Biol. 21:71-75.
Yonzon, C. R., Jeoung, E., Zou, S., Schatz, G. C., Mrksich, M., and Duyne, R. P. V. “A comparative analysis of localized and propagating surface plasmon resonance sensors: the binding of Concanavalin A toMonosaccharide functionalized self-assembled monolayer,” Journal of the American Chemical Society 126:12669-12676 (2005).
Zhang, Y., Hammer, D. A. and Graves, D. J. “Competitive hybridization kinetics reveals unexpected behavior patterns” Biophysical Journal 89:2950-2959 (2005).
Zhou, Y., Laybourn, P. J., Magill, J. V. and L'Rue, R. M. D. “An evanescent fluorescence biosensor using ionexchanged buried waveguides and the enhancement of peak fluorescence,” Biosensors and Bioelectronics 6:595-607 (1991).

Although the invention has been described with reference to the presently preferred embodiments, it should be understood that various modifications can be made without departing from the spirit of the invention. Accordingly, the invention is limited only by the following claims.

We claim:

1. A method of determining sequence similarity of a target nucleic acid and a probe, the method comprising:
a) bringing into contact the target nucleic acid, the probe, and a labeled competitor nucleic acid;
b) simultaneously incubating the target nucleic acid, a probe, and the labeled competitor nucleic acid under conditions suitable for hybridization;
c) determining the binding pattern of the competitor nucleic acid to the probe in the presence of the target nucleic acid; and
d) determining sequence similarity of the target nucleic acid and the probe based on the results of step c).

2. A method of detecting the presence of a target nucleic acid in a sample, the method comprising:
a) simultaneously bringing into contact the sample, a probe, and a labeled competitor nucleic acid;
b) incubating the sample, the probe, and the labeled competitor nucleic acid under conditions suitable for hybridization;
c) determining the binding pattern of the competitor nucleic acid to the probe in the presence of the sample; and
d) detecting the presence of the target nucleic acid based on the results of step c).

3. A method of quantifying a target nucleic acid, the method comprising:
a) simultaneously bringing into contact a target nucleic acid, a probe, and a labeled competitor nucleic acid;
b) incubating the target nucleic acid, the probe, and the labeled competitor nucleic acid under conditions suitable for hybridization;
c) determining the amount of labeled competitor nucleic acid bound to the nucleic acid probe; and
d) quantifying the target nucleic acid based on the results of step c)

4. The method of claim 1, wherein the target nucleic acid is labeled.

5. The method of claim 4, wherein the target nucleic acid label is distinct from the competitor nucleic acid label.

6. The method of claim 1, wherein the labeled competitor nucleic acid is present in higher concentrations.

7. The method of claim 1, wherein step c) is determined in real time.

8. The method of claim 7, wherein the hybridization pattern differs as a function of time.

9. The method of claim 8, wherein the labeled competitor nucleic acid initially dominates hybridization, and is then displaced by the target nucleic acid.

10. The method of claim 9, wherein the target nucleic acid and the probe are complementary in sequence.

11. The method of claim 7, wherein the target nucleic acid and the probe are not complementary in sequence.

12. The method of claim 11, wherein the target nucleic acid and the probe differ in sequence by one nucleotide.

13. The method of claim 9, wherein the labeled competitor nucleic acid and the probe are complementary in sequence

14. The method of claim 9, wherein the labeled competitor nucleic acid and the probe are not complementary in sequence.

15. The method of claim 14, wherein the labeled competitor nucleic acid and the probe differ by one nucleotide.

16. The method of claim 1, wherein the probe is attached to a solid support.

17. The method of claim 16, wherein the solid support is a microarray.

18. The method of claim 17, wherein more than one probe nucleic acid is used.

19. The method of claim 16, wherein said solid support is selected from the group consisting of glass and plastic.

20. The method of claim 1, wherein the target nucleic acid comprises a single nucleotide polymorphism (SNP).

21. The method of claim 1, wherein the labeled competitor nucleic acid molecule is fluorescently labeled.

22. The method of claim 1, wherein more than one labeled competitor nucleic acid is used.

23. The method of claim 22, wherein each labeled competitor nucleic acid is labeled with a different label.

24. The method of claim 1, wherein the probe is selected from the group consisting of DNA, RNA, or a combination thereof.

25. The method of claim 17, wherein the probes are attached to the microarray by a spacer.

26. The method of any one claim 1, wherein the target nucleic acid is selected from the group consisting of DNA, RNA, a peptide nucleic acid, or a combination thereof.

27. The method of claim 26, wherein the target DNA is cDNA.

28. The method of claim 26, wherein the DNA is mtDNA.

29. The method of claim 26, wherein the target RNA is mRNA.

30. The method of claim 26, wherein the target RNA is miRNA.

31. The method of claim 1, wherein the target is not amplified before being put in contact with the probe and the labeled competitor nucleic acid.

32. The method of claim 1, wherein the target is amplified before being put in contact with the probe and the labeled competitor nucleic acid.

33. The method of claim 1, wherein the labeled competitor nucleic acid is selected from the group consisting of DNA, RNA, a peptide nucleic acid, or a combination thereof.

35. The method of claim 1, further comprising curve fitting of the binding pattern of the labeled nucleic acid, thereby determining the sequence of the target nucleic acid.

36. The method of claim 35, wherein curve fitting of the binding patterns of the labeled nucleic acid to two or more probe sequences is used to determine the sequence of the target nucleic acid.

37. The method of claim 35, wherein curve fitting of the binding patterns of multiple competitor nucleic acid sequences is used determine the sequence similarity of the target nucleic acid and the probe

38. The method of claims 37, wherein curve fitting of the binding patterns of multiple competitor nucleic acid sequences is used to determine the sequences of two or more target nucleic acid sequences

40. The methods of claim 36, wherein the effects of binding of additional non-target nucleic acid sequences on the binding pattern of the competitor nucleic acid sequences is used to determine the sequence of the target nucleic acid.

41. The methods claim 36, wherein curve fitting of the binding patterns of the competitor nucleic acid and target nucleic acid sequences are used to determine the sequence of the target nucleic acid.

42. The method of claim 41, wherein the effects of binding of additional non-target nucleic acid sequences on the binding patterns of the competitor nucleic acid and target nucleic acid sequences is taken into account in determining the sequence of the target nucleic acid.

43. The method of claim 1, wherein determining the binding pattern of the competitor nucleic acid to the probe in the presence of the target nucleic acid occurs using real time signal acquisition.

44. The method of claim 43, wherein the real time signal acquisition is obtained using fluorescent resonance energy transfer (FRET).

45. The method of claim 43, wherein the real time signal acquisition is obtained using TIRF or evanescent wave excitation.

46. The method of claim 45, wherein evanescent wave excitation is accomplished via an optical waveguide.

47. The method of claim 46, wherein said optical waveguide is substantially planar.

48. The method of claim 47, wherein said substantially planar optical waveguide comprises thin dielectric or metallic films.

49. The method of claim 1, wherein the labeled competitor nucleic acid is labeled with nanoparticle scattering labels.

50. The method of claim 1, wherein the labeled competitor nucleic acid is labeled with Raman scattering labels.


SEQUENCE LISTING (8 sequences)

SEQ ID NO: 1 - DNA, 20 bp, Artificial Sequence
  Description of Artificial Sequence; note = synthetic construct
cgagggcagcaatagtacac

SEQ ID NO: 2 - DNA, 20 bp, Artificial Sequence
  Description of Artificial Sequence; note = synthetic construct
cgagggcagcattagtacac

SEQ ID NO: 3 - DNA, 61 bp, Artificial Sequence
  Description of Artificial Sequence; note = synthetic construct
gcaaactcaaactacgaacgcactcacagtcgcatcataatcctctctcaaggacttcaaa

SEQ ID NO: 4 - DNA, 61 bp, Artificial Sequence
  Description of Artificial Sequence; note = synthetic construct
gcaaactcaaactacgaacgcactcacagtcacatcataatcctctctcaaggacttcaaa

SEQ ID NO: 5 - DNA, 61 bp, Artificial Sequence
  Description of Artificial Sequence; note = synthetic construct
tttgaagtccttgagagaggattatgatgcgactgtgagtgcgttcgtagtttgagtttgc

SEQ ID NO: 6 - DNA, 223 bp, Artificial Sequence
  Description of Artificial Sequence; note = synthetic construct
catccaaaccccctgaagcttcaccggcgcagtcattctcataatcgcccacggactcacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactg

SEQ ID NO: 7 - DNA, 20 bp, Artificial Sequence
  Description of Artificial Sequence; note = synthetic construct
cgagggcagcataagtacac

SEQ ID NO: 8 - DNA, 20 bp, Artificial Sequence
  Description of Artificial Sequence; note = synthetic construct
cgagggcagcagtacacttt